(19) United States
(12) Patent Application Publication
(10) Pub. No.: US 2023/0277244 A1
(43) Pub. Date: Sep. 7, 2023

(54) METHOD AND SYSTEM FOR ESTIMATING DISTANCE BETWEEN A FIBER END AND A TARGET

(71) Applicant: Lumenis Ltd., Yokneam Ilit (IL)

(72) Inventor: Asaf Granot, Lotem (IL)

(73) Assignee: Lumenis Ltd., Yokneam Ilit (IL)

(21) Appl. No.: 18/196,828

(22) Filed: May 12, 2023

Related U.S. Application Data
(63) Continuation-in-part of application No. 17/535,172, filed on Nov. 24, 2021.
(60) Provisional application No. 63/341,654, filed on May 13, 2022.
(60) Provisional application No. 63/118,857, filed on Nov. 27, 2020.
(60) Provisional application No. 63/118,117, filed on Nov. 25, 2020.
(60) Provisional application No. 63/252,830, filed on Oct. 6, 2021.

(51) Int. Cl. A61B 18/22; G02B 23/24

(52) U.S. Cl. CPC A61B 18/22; G02B 23/2469; G02B 23/2423; A61B 2018/00642

(57) ABSTRACT

The present disclosure is related to field of Fiber Feedback (FFB) technology and provides a method and system for estimating the distance between a fiber end and a target. The method includes illuminating, by a Light Emitting, Transmitting and Detecting (LETD) system, the target with laser light of different wavelengths having low and high-water absorption coefficients, using different laser light sources, as well as receiving a returned signal corresponding to the incident laser light of different wavelengths, and detecting the returned signal to measure intensity values of the returned signal of a specific wavelength. Using the measured intensity values, a processing unit may estimate distance between the fiber end and the target. The present disclosure enables accurate estimation of distance between a fiber end and the target. The present disclosure also provides a robust distance estimation technique which is compatible with different types of targets.

### CROSS-REFERENCE TO RELATED APPLICATIONS

This application claims the benefit of and priority to U.S. Provisional Application No. 63/341,654, titled “Method and System for Estimating Distance Between a Fiber End and a Target”, filed on May 13, 2022, and is a continuation-in-part of, and claims the benefit of and priority to U.S. patent application Ser. No. 17/535,172, titled “Method and System for Estimating Distance Between a Fiber End and a Target”, filed on Nov. 24, 2021, which claims the benefit of and priority to U.S. Provisional Application No. 63/118,857, titled “Method and System for Estimating Distance Between a Fiber End and a Target”, filed on Nov. 27, 2020, U.S. Provisional Patent Application No. 63/118,117, titled “Apparatus and Method for Enhancing Laser Beam Efficacy in a Liquid Medium”, filed on Nov. 25, 2020, and U.S. Provisional Patent Application No. 63/252,830, titled “Method and System for Estimating Distance Between a Fiber End and a Target”, filed on Oct. 6, 2021, the entirety of which are incorporated herein by reference.

### TECHNICAL FIELD

The present disclosure generally relates to optical systems and optical fibers used in medical or therapeutic laser treatments. Particularly, but not exclusively, the present disclosure relates to a method and system for estimating the distance between a fiber end and a target.

### BACKGROUND

Introduction of lasers into the medical field and the development of fiber optic technologies that use lasers has opened numerous applications in treatments, diagnostics, therapies, and the like. Such applications range from invasive and non-invasive treatments to endoscopic surgeries and image diagnostics. For instance, in urinary stone treatment, the stones are required to be fragmented into smaller pieces. A technology known as laser lithotripsy may be used for such fragmenting processes, wherein for small to medium sized urinary stones, a rigid or flexible ureteroscope is placed through the urinary tract for illumination and imaging. Simultaneously, an optical fiber is inserted through a working channel of the ureteroscope, to a target location (e.g., to the location where the stone is present in the bladder, ureter, or kidney). The laser is then activated to fragment the stone into smaller pieces or to dust it. In another instance, a laser and optic fiber technology is used in coagulation or ablation treatments. During an ablation treatment, laser light is delivered to the tissue to vaporize the tissue. During a coagulation treatment, laser light is used to induce thermal damage within the tissue. Such ablation treatments may be used for treating various clinical conditions, such as Benign Prostate Hyperplasia (BPH), cancers such as prostate cancer, liver cancer, lung cancer and the like, and for treating cardiac conditions by ablating and/or coagulating a part of the tissue in the heart.

These treatments which use laser and optic fiber technology require high amounts of accuracy to ensure that the laser is aimed at the right target (stone, tissue, tumor etc.), to achieve the clinical objective of tissue ablation, coagulation, stone fragmentation, dusting and the like. Accordingly, it is important to know the distance between the target and end of the optical fiber (distal end) where the laser light is emitted, since the laser treatment parameters, such as energy, pulse width, laser power modulation, and/or repetition rate, are often determined based on the distance between the tip of the optical fiber to the target.

One of the existing techniques to estimate the distance between the distal end of an optical fiber and a target provides for measuring and comparing intensity values of reflections of the light beams, where the light beams are transmitted through the optical fiber by modulating the numerical apertures of the light beams. However, it is not always convenient to shift the numerical apertures of the light beams. Moreover, separation of the reflection of light beams of different numerical apertures, required for these techniques is difficult.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

An embodiment includes a system, comprising: a first laser source to generate laser light of a first wavelength; a second laser source to generate laser light of a second wavelength; an optical fiber having a distal end and a proximal end, the optical fiber configured to receive laser light from the first and second laser sources at the proximal end, to reflect a portion of the laser light from the proximal end, to emit a portion of the laser light out of the distal end, and to receive reflected laser light into the distal end; a first light detector to measure intensity of the reflected light; a second light detector; a mirror to direct, to the second light detector, the portion of the laser light reflected from the proximal end, the second light detector to measure intensity of the portion of the laser light reflected from the proximal end; and a processor and memory comprising instructions that when executed by the processor cause the processor to estimate a distance between the distal end of the optical fiber and a target based on the intensity of the reflected light measured by the first light detector and the intensity of the portion of the laser light reflected from the proximal end measured by the second light detector.

The system can include, wherein the first wavelength has a first water absorption coefficient higher than a second water absorption coefficient of the second wavelength.

The system can include, wherein the ratio of the first water absorption coefficient to the second water absorption coefficient is at least 2 to 1.

The system can include, wherein the first wavelength is approximately 1330 nm to approximately 1380 nm.

The system can include, wherein the second wavelength is approximately 1260 nm to approximately 1320 nm.

The system can include, comprising a third laser source to generate laser light of a third wavelength utilized to characterize a condition of the optical fiber, wherein the third wavelength has a third water absorption coefficient higher than the first and the second water absorption coefficients.

The system can include, wherein the third wavelength comprises approximately 1435 nm, approximately 2100 nm, or a wavelength between approximately 1870 nm and approximately 2050 nm.

The system can include, wherein the light detector measures a first intensity value of the reflected light corresponding to the laser light of the first wavelength and a second intensity value of the reflected light corresponding to the laser light of the second wavelength.

The system can include, wherein the instructions, when executed by the processor, further cause the processor to: compute a ratio of the first intensity value and the second intensity value; and estimate the distance between the distal end of the optical fiber and the target based on the ratio of the first intensity value and the second intensity value.

The system can include, wherein one or more of the first and second laser sources comprise a polarization maintaining pigtailed fiber laser, a single mode pigtailed fiber laser, or a free space laser.

The system can include, comprising a wave division multiplexer (WDM) coupled to a proximal end of the optical fiber, the WDM to arrange the laser light of the first wavelength and the laser light of the second wavelength to enter a proximal end of the optical fiber at one or more of a same point and a same angle.

An embodiment includes a method, comprising: illuminating a target with laser light of a plurality of different wavelengths; receiving first reflected light beams from the target via an optical fiber; receiving second reflected light beams from a proximal end of the optical fiber; measuring intensity of the first and second reflected light beams with a plurality of light detectors; and estimating a distance between a distal end of the optical fiber and the target based on intensity of the reflected light beams measured with the one or more light detectors.

The method can include, comprising emitting the laser light of the plurality of different wavelengths via the optical fiber to illuminate the target.

The method can include, comprising measuring a first intensity value of the reflected light beams corresponding to laser light of a first wavelength and a second intensity value of the reflected light beams corresponding to laser light of a second wavelength.

The method can include, computing a ratio of the first intensity value and the second intensity value; and estimating the distance between the distal end of the optical fiber and the target based on the ratio of the first intensity value and the second intensity value.

An embodiment includes at least one non-transitory computer-readable medium comprising a set of instructions that, in response to being executed by a processor circuit, cause the processor circuit to: determine a first intensity values based on first reflected laser light corresponding to a laser source, wherein the first reflected laser light enters a proximal end of an optical fiber, exits a distal end of the optical fiber, reflects of a target, and enters the distal end of the optical fiber; determine a second intensity value based on second reflected laser light corresponding to the laser source, wherein the second reflected laser light is reflected of the proximal end of the optical fiber; compute a ratio of the first intensity value and the second intensity value; and estimate a distance between the distal end of the optical fiber and the target based on the ratio of the first intensity value and the second intensity value.

The at least one non-transitory computer-readable medium can include, wherein the set of instructions, in response to execution by the processor circuit, further cause the processor circuit to subtract a first internal reflection value from a first measured intensity value to determine the first intensity value and subtract a second internal reflection value from a second measured intensity value to determine the second intensity value.

The at least one non-transitory computer-readable medium can include, wherein the set of instructions, in response to execution by the processor circuit, further cause the processor circuit to determine an internal reflection value based on third reflected laser light corresponding to laser light of a third wavelength, wherein the laser light of the third wavelength exits a laser source and the at least a portion of the third reflected laser light is reflected by a distal end of the optical fiber.

The at least one non-transitory computer-readable medium can include, wherein the set of instructions, in response to execution by the processor circuit, further cause the processor circuit to: compare the internal reflection value to a baseline internal reflection value; and adjust an operating parameter of a treatment beam based on comparison of the internal reflection value to the baseline internal reflection value.

The at least one non-transitory computer-readable medium can include, wherein the set of instructions, in response to execution by the processor circuit, further cause the processor circuit to: compare the internal reflection value to a baseline internal reflection value; characterize a condition of the optical fiber based on comparison of the internal reflection value to the baseline internal reflection value; and communicate an indication of the condition of the optical fiber via a user interface.

The at least one non-transitory computer-readable medium can include, wherein the set of instructions, in response to execution by the processor circuit, further cause the processor circuit to communicate an indication of the distance estimated between the distal end of the optical fiber and the target via a user interface.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system in accordance with one embodiment.

FIG. 2 illustrates a fiber optic cable in accordance with one embodiment.

FIG. 3 illustrates an LETD system in accordance with one embodiment.

FIG. 4 illustrates another LETD system in accordance with one embodiment.

FIG. 5 illustrates another LETD system in accordance with one embodiment.

FIG. 6 illustrates another LETD system in accordance with one embodiment.

FIG. 7 illustrates another LETD system in accordance with one embodiment.

FIG. 8 illustrates another LETD system in accordance with one embodiment.

FIG. 9 illustrates another LETD system in accordance with one embodiment.

FIG. 10 illustrates another LETD system in accordance with one embodiment.

FIG. 11 illustrates another LETD system in accordance with one embodiment.

FIG. 12 illustrates a method in accordance with one embodiment.

FIG. 13 illustrates another method in accordance with one embodiment.

FIG. 14 illustrates another method in accordance with one embodiment.

FIG. 15 illustrates another LETD system in accordance with one embodiment.

FIG. 16 illustrates another method in accordance with one embodiment.

FIG. 17 illustrates another LETD system in accordance with one embodiment.

FIG. 18 illustrates a computer-readable storage medium in accordance with one embodiment.

FIG. 19 illustrates another system in accordance with one embodiment.

### DETAILED DESCRIPTION

The present disclosure provides a method and system for estimating the distance between an optical fiber end and a target. It is to be appreciated that the efficiency of treatments using lasers often depend upon the relative position and orientation of the optical fiber tip with respect to the target. However, due to various factors such as movement of the optical fiber with respect to position and orientation within the body of a subject (for instance, a patient), tissue environment, movement of the tissue, surface of the target, color of the target, pigment of the target, optical fiber tip degradation during a treatment, water irrigation, and turbid environment (e.g., due to dusting), and the like, it is extremely difficult to determine or estimate the distance between the optical fiber tip and the target. Determining the distance between the optical fiber tip and the target is further complicated by the fact that the optical fiber tip is typically inserted into the body of the subject.

Incorrect estimation of the distance between the fiber end and the target and incorrect estimation of the orientation of the fiber end can lead to aiming the laser at a region which is not the region of interest of the target. This may lead to unnecessary complications, and in some cases, it can also lead to permanent damage to certain parts of the tissues, organs, etcetera of the subject, which could make portions of the body the subject dysfunctional. In some other scenarios, incorrect distance measurement and orientation may lead to an increase in the duration of the treatment, or may lead to low quality ablation/fragmentation results. In some cases, such as BPH or cancer, if the tumor is not ablated properly, it may lead to regrowth of the tumor (or other undesired tissue) leading to further complications. Therefore, it is important to determine an accurate (or maintain a desired) distance between the optical fiber tip and the target while performing certain treatments using laser and optical fiber technology as discussed above.

The method includes illuminating, by a light emitting, transmitting and detecting (LETD) system, a target with laser light of different wavelengths having low and high-water absorption coefficients, using different laser light sources. The wavelengths may be selected in such a way that, they are close to each other and belong to the same “nm scale.” Further, the LETD system receives returned signals corresponding to the incident laser light of different wavelengths. The returned signals comprise light beams reflected from the target post illumination. The one or more light detectors configured in the LETD system may detect the returned signals to measure intensity values of the returned signals of a specific wavelength. Using the measured intensity values, a processing unit may then estimate the distance between the fiber end and the target.

The present disclosure uses the described LETD system in different configurations comprising different arrangements of various optical components, such as beam combiners, beam splitters, polarizers, collimators, wave division multiplexers (WDM), light detectors and the like. The present disclosure enables accurate estimation of the distance between a fiber end and a target. Additionally, the present disclosure provides a robust distance estimation technique that is compatible with different types of targets. Further, the present disclosure may be used for the purpose of controlling and/or adjusting one or more operational parameters. For instance, during a treatment, the target may move around, back, and forth or otherwise, or may change one or more of its shape, size, composition, pigment, and color. Therefore, parameters for the laser sources that are pre-set before initiating lasing on the target, may become less effective. Conventionally, such pre-set parameters are manually changed, which may be error prone and time consuming, or in some cases the pre-set parameters may be left unchanged which may lead to scenarios where the optical fiber may be too close or too far from the target. Therefore, the present disclosure allows automatic and real-time monitoring of the distance between the optical fiber end and the target, and further enables automatically changing of the pre-set lasing parameters to adjust the lasing in accordance with the target shape, position etc. and to provide a higher likelihood of achieving the desired result or outcome from the treatment.

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

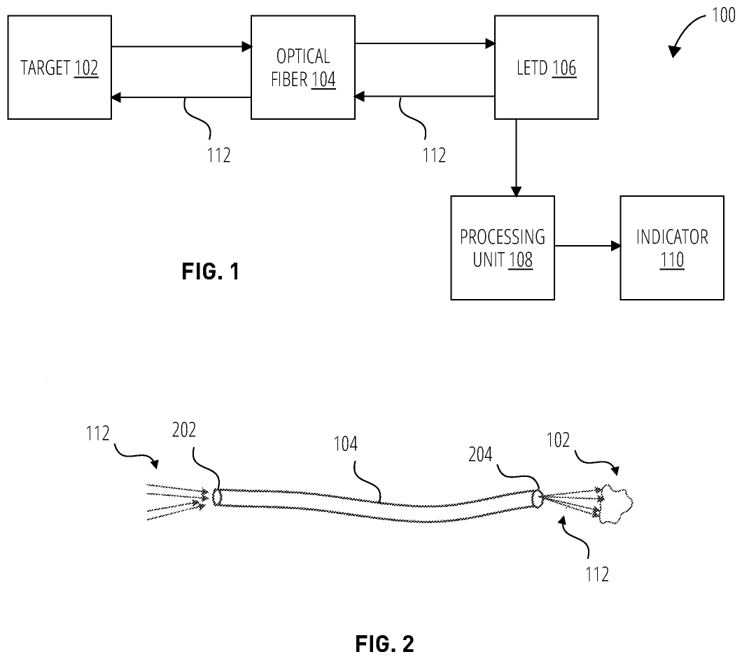

FIG. 1 show an exemplary system 100 for estimating a distance between a fiber end and a target in accordance with some embodiments of the present disclosure. In some embodiments, the exemplary system 100 comprises a target 102, an optical fiber 104, a light emitting, transmitting, detecting (LETD) system 106, a processing unit 108, and an indicator 110. The target 102 may be a tissue, a stone, a tumor, a cyst, and the like, within a subject, which is to be treated, ablated, or destroyed. In some embodiments, the subject may be a human or an animal.

Further, as depicted more fully in FIG. 2, the optical fiber 104 comprises a proximal end 202 and a distal end 204. The proximal end 202 is the end of the optical fiber 104 through which light beams 112 enter while the distal end 204 is the end of the optical fiber 104 through which light beams 112 are emitted and via which light beams 112 can be directed onto the target 102. For example, this figure depicts light beams 112 entering the optical fiber 104 at the proximal end 202, propagating through length of the optical fiber 104, exiting the optical fiber 104 at the distal end 204, and being incident on the target 102 from the distal end 204 of the optical fiber 104.

Returning to FIG. 1, the light beams may be beams directed from a light source (e.g., included in LETD system 106, or the like). The light source can be a laser light source. As an example, such a laser light sources may include, but is not limited to, solid-state lasers, gas lasers, diode lasers, and fiber lasers. The light beams 112 may include one or more of an aiming beam, a treatment beam, and any other beam transmitted through the optical fiber 104.

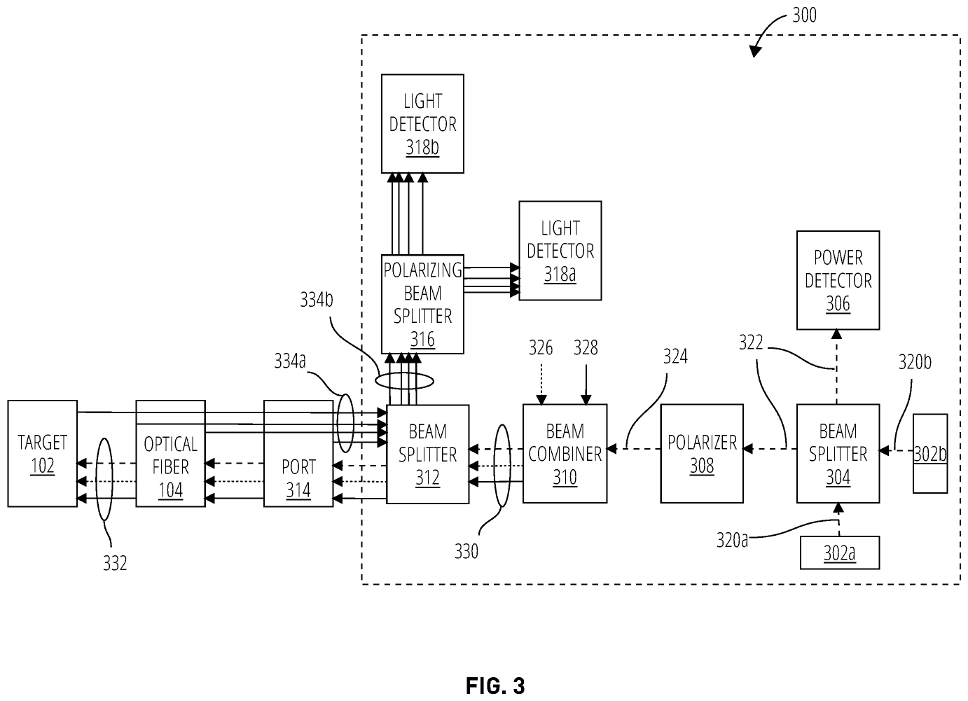

In various embodiments, an aiming beam may include a light beam of low intensity that is transmitted through the optical fiber 104 to estimate the distance between the optical fiber end (e.g., the distal end 204) and the target 102. In several embodiments, a treatment beam may include a light beam of high intensity that is transmitted through the optical fiber 104 to treat the target 102. In some embodiments, the different light beams may be produced by one or more laser light sources. As a specific example, the aiming beam may be generated by one laser source and the treatment beam may be generated by another laser source. In another example, both the aiming beam and the treatment beam may be generated by a single laser source. With yet another example, different laser light sources may be used to generate light beams of different wavelengths, characteristics, and the like. This is described in greater detail below, for example, with respect to FIG. 3 to fig.

[symbol]

The optical fiber 104 may be in optical communication with the LETD system 106 as shown in the FIG. 1 and arranged to receive light beams, to be aimed at the target 102, and to deliver reflected light beams that reflect from the surface of and region around the target 102. In some embodiments, the optical fiber 104 may be optically, mechanically, and/or electrically coupled with the LETD system 106 via a port (shown in other figures herein).

In some embodiments, the LETD system 106 comprises optical components which may include, but are not limited to, one or more of laser light sources, polarizers, beam splitters, beam combiners, light detector, wavelength division multiplexers, collimators, circulators, that are configured in various combinations, as explained in detail in further parts of the present disclosure.

In many embodiments, laser light sources are configured to generate laser light beams, such as a low intensity aiming beam for the purpose of aiming the light beams 112 at the target 102 and a high intensity treatment beam for treating the target 102, and/or light beams with varying characteristics (e.g., intensities, wavelengths, etcetera) based on the application. Each laser light source may be configured to generate laser light having different wavelengths, where each of the different wavelengths can have different water absorption coefficients. Further, each laser light source may have the same aperture or different apertures. In some embodiments, each laser light source may be designated with a different purpose, for instance, one laser light source may be configured to generate aiming beams of a particular intensity and one laser light source may be configured to generate a treatment beam of a particular intensity, and one or more laser light sources may be configured to generate light beams of a specific wavelength having specific water absorption coefficient. Additionally, each laser light source may be configured to generate polarized laser light or unpolarized/depolarized light.

Polarizers may include the optical components that act as an optical filter. For example, polarizers may be configured to allow light beams of a specific polarization to pass through, and to block the light beams of different polarizations. Therefore, when undefined light (or light beams of mixed polarity) are provided as input to a polarizer, the polarizer provides a well-defined single polarized light beam as an output.

Beam splitters may include the optical components used to split incident light at a designated ratio into two separate beams. Further, beam splitters may be arranged to manipulate light to be incident at a desired angle of incidence (AOI). Therefore, in many embodiments, a beam splitter can be primarily configured with two parameters, a ratio of separation and an AOI. The ratio of separation comprises the ratio of reflection to transmission (reflection/transmission (R/T) ratio) of the beam splitter. Accordingly, as used herein, if the ratio of separation for a beam splitter is indicated as 50:50, it means that the beam splitter splits the incident light beams in a R/T ratio of 50:50. In other words, the beam splitter splits the incident light beams by changing the incident light by reflecting 50 percent and transmitting the other 50 percent. Further, as an example, if the AOI for the beam splitter is indicated as 45 degrees, it means that the beam splitter ensures that the light beams would be incident at an angle of 45 degrees. Beam splitters may include, but are not limited to, polarizing beam splitters and non-polarizing beam splitters. Polarizing beam splitters may split incident light based on the S-polarization component and P-polarization component, such as, for example by reflecting the S-polarized component of light and transmitting the P-polarized component of light (or vice-versa). In some embodiments, non-polarizing beam splitters may split incident light beams based on a specific R/T ratio while maintaining the original polarization state of the incident light beams.

Beam combiners may include partial reflectors that combine two or more wavelengths of light, such as by using the principle of transmission and reflection as explained above. In many embodiments, a beam combiner may be a combination of beam splitters and mirrors, which perform the functionality of combining light of two or more wavelengths.

Light detectors may include devices that detect and/or measure characteristics of light beams and encode the detected and/or measured characteristics in electrical signals. For example, light detectors may detect the specific type of light beams (as preconfigured), and convert the light energy associated with the detected light beams into electrical signals. In some embodiments, wavelength division multiplexing may include a technology that combines a number of optical carrier signals onto a single optical fiber while using laser lights of different wavelengths.

A collimator may include a device that narrows down light beams. To narrow down the light beam, a collimator may be configured to cause the directions of motion to become more aligned in a specific direction (for example, parallel rays), or to cause the spatial cross section of the beam to become smaller. In many embodiments, a collimator may be used to change diverging light from a point source into a parallel beam.

A circulator may include a multi-port optical device configured to receive and emit light via a predetermined sequence of the multiple ports. For example, a circulator may include a three (or four, or five, etc.) port optical device designed such that, light entering any one port exits from the next port. In one such example, light entering a first port may exit a second port, light entering the second port may exit a third port, and light entering the third port may exit the first port. Oftentimes circulators may be utilized to allow light beams to travel in only one direction.

It is noted that where optical component described herein list specific parameters, such as, a beam splitter having an R/T ratio of 50:50 and an AOI of 45 degrees, these parameters are provided for general understanding of the concepts disclosed and not to be limiting. As a specific example, a beam splitter could be provided in various embodiments described herein having a different R/T ratio and/or AOI than specified here without departing from the scope of the disclosure and claims. In one such example, an AOI of 40 degrees may be utilized. In another such example an R/T ratio of 47:53 may be utilized.

The LETD system 106 is further associated with a processing unit 108 and/or a communication network (not shown). In some embodiments, the communication network may be a wired communication network or a wireless communication network. The processing unit 108 may be configured to receive measured values from the LETD system 106 and estimate the distance between the distal end 204 of the optical fiber 104 and the target 102 based on the measured values. In some embodiments, the processing unit 108 may be a standalone device with the processing capability required for distance estimation. For example, processing unit 108 can include circuitry arranged to determine a distance based on electrical signals received from the LETD system 106. As another example, processing unit 108 can include circuitry and memory comprising instructions, which when executed by the circuitry cause the circuitry to determine a distance based on electrical signals received from the LETD system 106. Still, in some other embodiments, the processing unit 108 may be a computing device such as a laptop, a desktop, a mobile phone, a tablet phone, and the like, configured to perform the distance estimation using their processing capability.

The processing unit 108 may be associated with the indicator 110 to indicate the estimated distance between the distal end of the optical fiber 104 and the target 102. The indicator 110 may include, but is not limited to, a visual indicator which displays the estimated distance, an audio indicator which announces the estimated distance, or a haptic indicator which indicates the estimated distance via vibration patterns. In various embodiments, the indicator may be presented via a graphical user interface and/or overlaid on a graphical representation, such as a video feed. In some embodiments, the computing device configured as the processing unit 108 may be configured to perform the functionalities of the indicator 110. In some other embodiments, the indicator 110 may be a standalone device which is configured to indicate the estimated distance between the distal end of the optical fiber 104 and the target 102.

Various exemplary configurations for estimating distance between the fiber end and the target are explained in detail below. However, values and parameters associated with different optical components used in each of the below explained configurations, should be considered purely exemplary, and not be construed as a limitation of the present disclosure.

FIG. 3 illustrates a LETD system 300, which can be implemented as the LETD system 106 of system 100. The LETD system 300 can be configured to estimate distance between a fiber end and a target in accordance with some embodiments of the present disclosure. As depicted, LETD system 300 includes a number of laser sources. In particular, the laser source 302a and 302b are shown. With some embodiments, the laser source 302a and 302b can be polarized laser sources. Additionally, LETD system 300 includes a beam splitter 304, a power detector 306, and a polarizer 308. The laser source 302a and 302b are arranged to generate light beam 320a and 320b, respectively. With some embodiments, the laser source 302a is arranged to generate the light beam 320a having a first wavelength while the laser source 302b is arranged to generate the light beam 320b having a second wavelength different than the first wavelength wherein the first wavelength has an absorption coefficient (e.g., in water, or the like) higher than an absorption coefficient of the second wavelength.

As used herein, the light beam 320a generated by the laser source 302a can be referred to as high water absorption coefficient light (HI) while the light beam 320b generated by the laser source 302b can be referred to as low water absorption coefficient light (LO). It is to be appreciated that even though the terms “high” and “low” are used they are intended to be interpreted relative to each other, or in the alternative relative to a threshold characteristic describing the water absorption of a particular wavelength. For example, a high-water absorption characteristic can be greater than or equal to 50% while a low water absorption characteristic can be less than or equal to 50%.

In various embodiments, the ratio of the high-water absorption coefficient to the low absorption coefficient may be approximately 1:2. For example, laser light 225a may utilize a wavelength of approximately 1310 nm and have a water absorption coefficient of approximately 0.1651 while laser light 225b may utilize a wavelength of approximately 1340 nm and have a water absorption coefficient of approximately 0.333. The higher the ratio between the high and low absorption coefficients may result in less sensitivity to system noise (e.g., electrical or opto-mechanical noise), but the resulting system may not be effective at distances over 3 mm. The lower the ratio between the high and low absorption coefficients may result in higher sensitivity to system noise, but the resulting system may remain effective up to distances of 5 or 6 mm. In some examples, the laser source 302a and 302b may be polarization maintaining (PM) pigtailed fiber lasers.

The laser source 302a and 302b are associated with and in optical communication with the beam splitter 304. Said differently, the light beam 320a and 320b generated by laser source 302a and 302b, respectively, are provided as input to the beam splitter 304, which is configured to split the incident light beam 320a and 320b at a ratio of approximately 50:50 (e.g., 47:53 or 49:51), such that the incident light beam 320a and 320b align along a single optical path as light beams 322. However, it will be appreciated that any ratios between 99:1 and 1:99 may be utilized without departing from the scope of this disclosure. Similarly, although AOIs of 45 degrees may be described in embodiments, it will be appreciated that any AOIs between 1 and 89, such as 43-47 degrees, 40 degrees, or 20 degrees, may be utilized without departing from the scope of this disclosure.

The power detector 306 is associated with and in optical communication with the beam splitter 304. The power detector 306 is arranged to measure the optical power in the optical signal (e.g., the portion of the light beams 320a and 320b routed to the power detector 306) corresponding to each wavelength of light in the light beam 322. In some embodiments, the term “optical power” may refer to energy transported by a certain laser beam, per unit time.

The beam splitter 304 is further associated with an in optical communication with the polarizer 308. The beam splitter 304 is further arranged to provide a portion of the light beams 320a and 320b, denoted as light beam 322, which is aligned along a single optical path, as an input to the polarizer 308. In some embodiments, the polarity of the polarizer 308 may be pre-configured and arranged to output polarized light beam 324. The LETD system 300 further includes a beam combiner 310, which is in optical communication with the polarizer 308. In such a manner, the polarized light beam 324 obtained as an output from the polarizer 308 is provided as input to the beam combiner 310.

The beam combiner 310 may combine the polarized light beams 324 with a treatment beam 326 and an aiming beam 328. In some other embodiments, the treatment beam 326 may be generated by one or more laser sources (not shown) other than the laser sources 302a and 302b. As an example, the treatment beam 326 may be generated by a solid-state laser or a fiber laser, such as a holmium (HO) laser or a Thulium fiber laser (TFL). However, this should not be considered as a limitation of the present disclosure, since the treatment beam 326 may be generated by lasers other than HO or TLF, such as Neodymium, Erbium, and the like. In some other embodiments, the treatment beam 326 and the aiming beam 328 may be generated by the laser sources 302a and/or 302b.

The beam combiner 310 can combine the polarized light beam 324 with the treatment beam 326 and aiming beam 328 to form the combined light beam 330. The LETD system 300 further includes a beam splitter 312 and a port 314. The beam splitter 312 is arranged in optical communication with the beam combiner 310. The beam splitter 312 can receive the combined light beam 330, comprising the polarized light beam 324, treatment beam 326, and aiming beam 328. The beam splitter 312 may have a configuration of a 50:50 R/T ratio and a 45-degree AOI.

In such an arrangement, the beam splitter 312 can split the combined light beam 330 in the ratio of 50:50, such that, the polarized light beam 324, treatment beam 326, and aiming beam 328 are aligned along a single optical path. The beam splitter 312 is optically coupled to the optical fiber 104 (e.g., via the port 314, or the like) such that a portion of the combined light beam 330, which is the output of the beam splitter 312 is transmitted through the optical fiber 104 (e.g., via the port 314) and denoted as light beams 332. The light beams 332 are transmitted to the proximal end 202 of the optical fiber 104, which then propagate through the length of the optical fiber 104 to be delivered to the target 102 from the distal end 204 of the optical fiber 104. As an example, the target 102 may be a tissue, a stone, a tumor, a cyst, and the like, within a subject, which is to be treated, ablated, destroyed, or the like.

When the light beams 332 are delivered to the target 102 via the optical fiber 104, the target 102 may reflect some portion of the incident light beams 332 away from the optical fiber 104 and some portion of the light towards the optical fiber 104, wherein the portion of light reflected towards the optical fiber 104 may re-enter the optical fiber 104, at the distal end 204 of the optical fiber 104. The portion of the reflected light re-entering at the distal end may be referred as reflected light beams 334a. The reflected light beams 334a may be transmitted “backward” in the optical fiber 104 from the distal end 204 to the proximal end 202 of the optical fiber 104. When the reflected light beams 334a reaches the proximal end 202 of the optical fiber 104, the reflected light beams 334a may be subjected to the beam splitter 312. The reflected light beams 334a may include numerous reflections, such as from the target 102, the distal end 204 of the optical fiber 104, the proximal end 202 of the optical fiber 104, the port 314, and the like. As such, the reflected light beams 334a are no longer polarized.

The LETD system 300 further includes a polarizing beam splitter 316, a light detector 318a, and a light detector 318b. The reflected light beams 334a may be incident at an angle of 45 degrees to the beam splitter 312 and split in the ratio of 50:50 (or other ratio as detailed herein), resulting in the reflected light beams 334a being aligned along a single optical path as reflected light beams 334b. The polarizing beam splitter 316 is arranged in optical communication with the beam splitter 312 and arranged to receive the reflected light beams 334b and polarize the reflected light beams 334b. In some embodiments, the polarizing beam splitter 316 may split the reflected light beams 334b into reflected P-Polarized and transmitted S-polarized beams. One of the light detectors 318a or 318b may be configured to detect the P-polarized beams of the reflected light beams 334b while the other of the light detectors 318a or 318b may be configured to detect the S-polarized beams of the reflected light beams 334b. The light detectors 318a and 318b may measure intensities of the detected light beams of the reflected light beams 334b, respectively, and transmit the intensities to the processing unit 108. In some embodiments, the processing unit 108 may estimate distance between the distal end 204 of the optical fiber 104 and the target 102 based on the measured intensities. The method of estimating the distance between the distal end of the optical fiber 104 and the target 102 based on the measured intensities is explained in greater detail below.

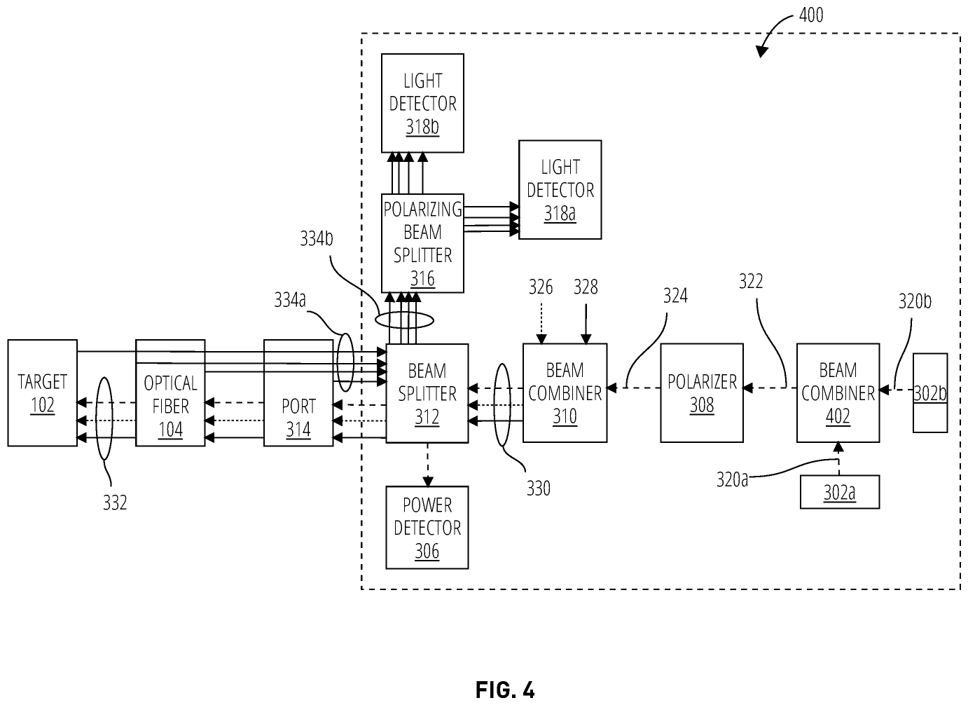

FIG. 4 illustrates a LETD system 400, which can be implemented as the LETD system 106 of system 100. The LETD system 400 can be configured to estimate distance between a fiber end and a target in accordance with some embodiments of the present disclosure. For convenience, where components of the LETD system 400 are the same as components of the prior described LETD systems (e.g., LETD system 300, or the like), the same reference numbers are used.

LETD system 400 is different than LETD system 300 in two constructional aspects. One of the constructional aspects which is different between LETD system 400 and LETD system 300 is the beam splitter 304 is replaced with a beam combiner 402. Since the beam splitter 304 is replaced with beam combiner 402, the power detector 306, which was associated with the beam splitter 304 in LETD system 300, is arranged to be associated with the beam splitter 312 in LETD system 400. Embodiments are not limited in this context.

LETD system 400 may include one or more polarized lasers, one or more beam splitters, a polarizer, one or more beam combiners, and one or more light detectors. The one or more beam splitters may be polarized beam splitters, non-polarized beam splitters, or a combination of both polarized and non-polarized beam splitters. As shown in FIG. 4, the LETD system 400 includes the laser source 302a, the laser source 302b, the beam combiner 402, the polarizer 308, the beam combiner 310, the beam splitter 312, the power detector 306, the polarizing beam splitter 316, the light detector 318a, and the light detector 318b. In this configuration, as shown in the FIG. 4, the laser source 302a outputs a light beam 320a with a wavelength having a high-water absorption coefficient (HI) and the laser source 302b outputs a light beam 320b with a wavelength having a low water absorption coefficient (LO).

The incident light beams from laser sources laser sources 302a and 302b are provided as input to the beam combiner 402, which is configured to combine the incident light beams 320a and 320b that are generated by the laser sources 302a and 302b into light beam 322. Further, the output of the beam combiner 402 (e.g., light beam 322) can be provided as an input to the polarizer 308 for providing the polarized light beam 324 as an output. In some embodiments, the polarization of the polarizer 308 may be pre-configured. Thereafter, the polarized light beam 324 obtained as an output from the polarizer 308 may be provided as input to the beam combiner 310. The beam combiner 310 may combine the polarized light beams 324 with the aiming beam 328 and the treatment beam 326 into combined light beam 330, as shown in the FIG. 4.

The combined light beam 330 comprising the aiming beam 328, the treatment beam 326, and the polarized light beams 324 from laser sources 302a and 302b, may be subjected to the beam splitter 312 having a configuration of an R/T ratio of 50:50 and an AOI of 45-degree (or any other R/T ratio and AOI as outlined hereby). The beam splitter 312 may split the combined light beam 330 in the ratio of 50:50, such that, the aiming beam 328, the treatment beam 326, and the polarized light beams 324 may be aligned along a single optical path.

The power detector 306 associated with the beam splitter 312 may measure the power in the optical signal (the polarized light beam 324, or the like) corresponding to each wavelength. In various embodiments, the power detector 306 may detect cumulative energy of the optical signal received at the beam splitter 312. In some embodiments, the term “optical power” may refer to energy transported by a certain laser beam, per unit time. The light beams 332, which are the output of the beam splitter 312, are then transmitted to the optical fiber 104 (e.g., via a port 314) as outlined above with respect to FIG. 3. Additionally, reflected light beams 334a are received and processed as outlined above with respect to FIG. 3.

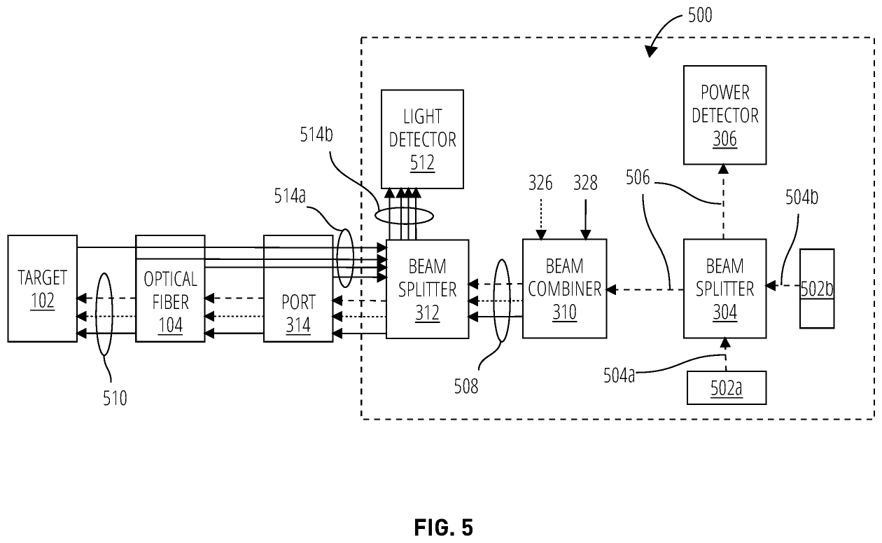

FIG. 5 shows a LETD system 500 for estimating distance between a fiber end and a target in accordance with some embodiments of the present disclosure. The present disclosure can work with polarized and non-polarized laser sources. Accordingly, in LETD system 500, the laser sources 502a and 502b used for providing incident light beams (source light) are non-polarized laser sources. As an example, the laser sources 502a and 502b may be Single Mode (SM) fiber pigtailed lasers. When the laser sources 502a and 502b are non-polarized laser sources, there is no requirement of the polarizer 308, the polarized polarizing beam splitter 316, the light detector 318a for detecting P-polarized light beams and the light detector 318b for detecting S-polarized light beams, as depicted in LETD system 300 and LETD system 400 described above.

LETD system 500 may include one or more non-polarized lasers, one or more beam splitters, a beam combiner, and a light detector. The one or more beam splitters may be non-polarized beam splitters. As shown in the FIG. 5, the LETD system 500 includes a non-polarized laser source 502a, a non-polarized laser source 502b, the beam splitter 304, the power detector 306, the beam combiner 310, the beam splitter 312, and a light detector 512.

Like the prior configurations, in LETD system 500, the non-polarized laser source 502a can have a wavelength with high water absorption coefficient (HI) while the non-polarized laser source 502b can have a wavelength with low water absorption coefficient (LO). The light beams 504a and 504b from laser sources 502a and 502b, respectively, are provided as input to the beam splitter 304 which is configured to split the incident light beams at a ratio of 50:50, in a way that, the incident light beams 504a and 504b align along a single optical path as light beams 506.

The power detector 306 associated with the beam splitter 304 may measure the power in the optical signal (light beam 506) corresponding to each wavelength. Since, LETD system 500 is implemented in a non-polarized environment, polarization based optical components, such as, a polarizer and a polarized beam splitter are not needed in this configuration. Therefore, the output of the beam splitter 304, which is the incident light beams 504a and 504b aligned along a single optical path as light beam 506, may be provided as an input to the beam combiner 310. The beam combiner 310 may combine the light beams 506 coming from the beam splitter 304 with the aiming beam 328 and the treatment beam 326, as shown in the FIG. 5.

In some embodiments, the aiming beam 328 and the treatment beam 326 may be generated by one or more laser sources other than the laser sources 502a and 502b. In some other embodiments, the aiming beam 328 and the treatment beam 326 may be generated by the laser sources 502a and 502b. The combined light beam 508 comprising the aiming beam 328, the treatment beam 326 and the non-polarized light beams light beam 506, may be subjected to beam splitter 312 having a configuration of ratio 50:50 and AOI of 45 degree (or any other R/T ratio and AOI as outlined hereby). The beam splitter 312 may split the combined light beam 508 in the ratio of 50:50, such that, the aiming beam 328, the treatment beam 326 and the non-polarized light beams light beam 506 are aligned along a single optical path. The light beams 510 which are the output of the beam splitter 312, are then transmitted to an optical fiber 104 (e.g., via a port 314) while reflected light beams 514a is transmitted backwards, as shown in the FIG. 5 and described above.

Since, LETD system 500 is implemented in a non-polarized environment, the reflected light beams 334a is only subjected to the second beam splitter 312 to align the optical path of the reflected light beams 514a while a polarized beam splitter, as depicted in LETD system 300 and LETD system 400 is not needed. The reflected light beams 514a would be incident at an angle of 45 degrees to the beam splitter 312 and split in the ratio of 50:50. The reflected light beams 334b that emerge out of the beam splitter 312 may be directly detected by a single detector. As such, LETD system 500 provides the light detector 512.

The light detector 512 may measure intensity of the detected light beams of the reflected light beams 514b, respectively, and transmit the intensity to the processing unit 108. In some embodiments, the processing unit 108 may estimate the distance between the distal end 204 of the optical fiber 104 and the target 102 based on the measured intensities. The method of estimating the distance between the distal end of the optical fiber 104 and the target 102 based on the measured intensities is explained in greater detail below.

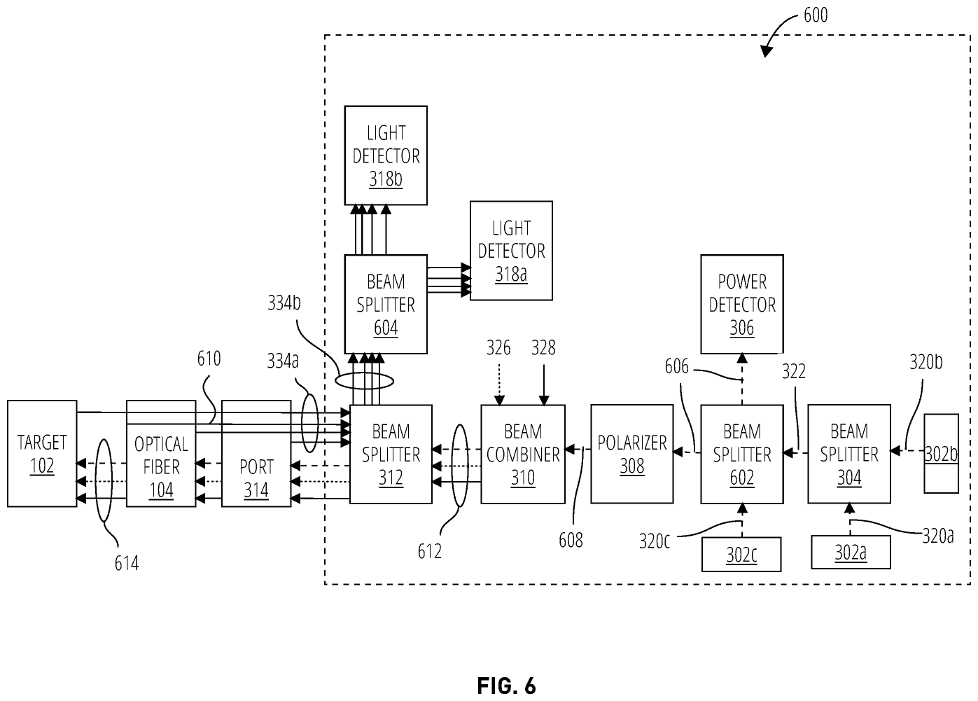

FIG. 6 shows an exemplary LETD system 600 for estimating distance between a fiber end and a target in accordance with some embodiments of the present disclosure. The LETD system 600 comprises a third polarized laser source 302c, which is introduced for the purpose of calibration of the optical fiber condition in real-time. As an example, the condition of the optical fiber 104 may include, but is not limited to, any changes or degradation of the distal or proximal ends of the optical fiber 104, fiber bending effects on polarization scrambling, or any other degradations and changes occurring in the optical fiber 104. Changes in condition of the optical fiber 104, specifically the tips/ends (e.g., the input and output facets) of the optical fiber 104 may adversely affect the transmitted and reflected light beams, causing large number of reflections, loss of energy and inaccurate measurements. This can affect the accuracy of the distance estimation, thereby leading to incorrect positioning of the optical fiber 104 during a treatment.

LETD system 600 may include one or more polarized lasers, one or more beam splitters, a polarizer, a beam combiner, and one or more light detectors. The one or more beam splitters may be polarized beam splitters, non-polarized beam splitters, or a combination of both polarized and non-polarized beam splitters. As shown in the FIG. 6, the LETD system 600 includes the polarized laser source 302a, the polarized laser source 302b, and the polarized laser source 302c, the beam splitter 304, the beam splitter 602, the power detector 306, the polarizer 308, the beam combiner 310, the beam splitter 312, the beam splitter 604, the light detector 318a, and the light detector 318b. As shown in the FIG. 6, the light beams 320a and 320b from laser sources 302a and 302b are provided as input to the beam splitter 304, which is configured to split the light beams 320a and 320b at a ratio of 50:50, such that the light beams 320a and 320b align along a single optical path forming light beam 322. Further, the output of the beam splitter 304, which is the light beams 320a and 320b aligned along a single optical path (e.g., light beams light beam 322) can be provided as an input to the beam splitter 602, which is also configured to split the incident light beams in the ratio of 50:50 forming polarized light beams 606 that comprise light beams 320a, 320b, and 320c.

At the beam splitter 602, incident light beams 320c from the polarized laser source 302c (e.g., light meant for calibration) are provided as input along with the output of the beam splitter 304 (e.g., light beams light beam 322). The power detector 306 associated with the beam splitter 602 may measure the power in the optical signal (e.g., light beam 606) corresponding to each wavelength arriving at the beam splitter 602. Along with the output of the beam splitter 304, the beam splitter 602 receives incident light beams from the polarized laser source 302c.

In some embodiments, the polarized laser source 302c has a wavelength with a very high-water absorption coefficient (e.g., substantially, completely, or almost completely, absorbed by water) relative to the wavelength of light emitted by the laser sources 302a and 302b. As an example, the wavelength of the polarized laser source 302c may be approximately 1435 nm and have a water absorption coefficient of approximately 31.55 (or approximately 100 times the “high” water absorption source). At a distance of 0.5 mm with a wavelength of 1435 nm about 98-99% of the light is absorbed. In some embodiments, the calibration light source may have a wavelength of approximately 1420 to approximately 1440 (resulting in a water absorption coefficient of approximately 30. Alternative, or additional, wavelengths with a very high-water absorption coefficient may be utilized (e.g., 1870-2070 nm). However, the further the wavelength is from the HI and LO wavelengths (e.g., 1310 nm and 1340 nm, respectively) may lead to a more complicated optical design. For example, a detector may cover range of approximately 1100 nm to 1600 nm, and if the very high-water absorption coefficient laser has a wavelength of 2000 nm, a unique, or additional, detector would be required. In some embodiments, the calibration laser may have a wavelength of approximately 1435 nm, approximately 2100 nm, or a wavelength between approximately 1870 nm and approximately 2050 nm.

Based on the readings of the polarized laser source 302c (e.g., as measured by the power detector 306) the processing unit 108 may define an optical baseline characteristic of the “quality” of fiber tip at the distal end 204 of the optical fiber 104. More specifically, as the laser source 302c is highly absorbed in water, light from the laser source 302c will not likely reach the target tissue, and as a result hardly any light from the laser source 302c will be reflected back into the optical fiber 104 as part of the reflected light beams 334a. Therefore, the component of reflected light beams 334a (e.g., light reflection 610) with the wavelength of light associated with the laser source 302c are mainly attributable to the optical characteristics of distal end 204 of the optical fiber 104. It is to be appreciated that the distal end 204 of the optical fiber 104 goes through degradation during a laser treatment due to, for example, heat and cavitation. In many embodiments, increased intensity readings of back light reflection 610 may indicate optical fiber tip degradation. In several embodiments, at a certain threshold of intensity changes from the baseline reading for a specific fiber (e.g., 10% to 50%, greater than or equal to 25%, 50%, 75%, 90%, between 10% and 100%, or the like) the processing unit 108 may indicate that the optical fiber 104 should be checked or replaced, such as through a user interface and/or audible alarm. In addition, optical fiber tip degradation may cause higher internal reflections from the distal end of the fiber, of light from polarized laser sources 302a and 302b. Whether or not the laser sources are polarized may have minimal effect on internal reflections because the light is randomly depolarized in the fiber. However, monitoring the reflections from the fiber distal end by the very high absorption coefficient laser (e.g., 1435 nm laser) can be utilized to determine changes in distal end reflections (in percentage of initial reflections of 1435 versus real-time reflections). Further, the changes in distal end reflections may applied on the initial reflections from the distal end for the LO laser (e.g., 1310 nm laser) and HI laser (e.g., 1340 nm laser) to update the initial reflections.

Moreover, fiber tip degradation may change the ratios between polarities P and S in reflected light beams 334a or light reflection 610. Therefore, creating baseline readings, for a specific optical fiber 104 currently in use, and monitoring these baselines on the fly, may allow more accurate distance estimations even when and during the tip of the fiber degrades and until degradation reaches a threshold level that indicates that the optical fiber 104 should be replaced. Further, output of the beam splitter 602, or light beam 606, which includes the light beams 320a, 320b, and 320c that are aligned along a single optical path, may be provided as an input to the polarizer 308 to obtain a single polarized light beam 608 as an output. In some embodiments, the polarization of the polarizer 308 may be pre-configured.

The polarized light beam 608 obtained as an output from the polarizer 308 can be provided as input to the beam combiner 310. The beam combiner 310 may combine the polarized light beam 608 with the aiming beam 328 and the treatment beam 326 to form combined light beam 612 as shown in FIG. 6. As detailed above, the aiming beam 328 and/or the treatment beam 326 may be generated by one or more laser sources other than the laser sources 302a, 302b, or 302c or the aiming beam 328 and/or the treatment beam 326 may be generated by these laser sources.

The combined light beam 612 comprising the aiming beam 328, the treatment beam 326 and the polarized light beams 608 may be subjected to the beam splitter 312 having a configuration of ratio 50:50 and a 45-degree AOI. The beam splitter 312 may split the combined light beam 612 in the ratio of 50:50, such that, the aiming beam 328, the treatment beam 326, and the polarized light beams 608 are aligned along a single optical path. The light beams 614, which are the output of the beam splitter 312, are then transmitted to optical fiber 104 (e.g., via port 314).

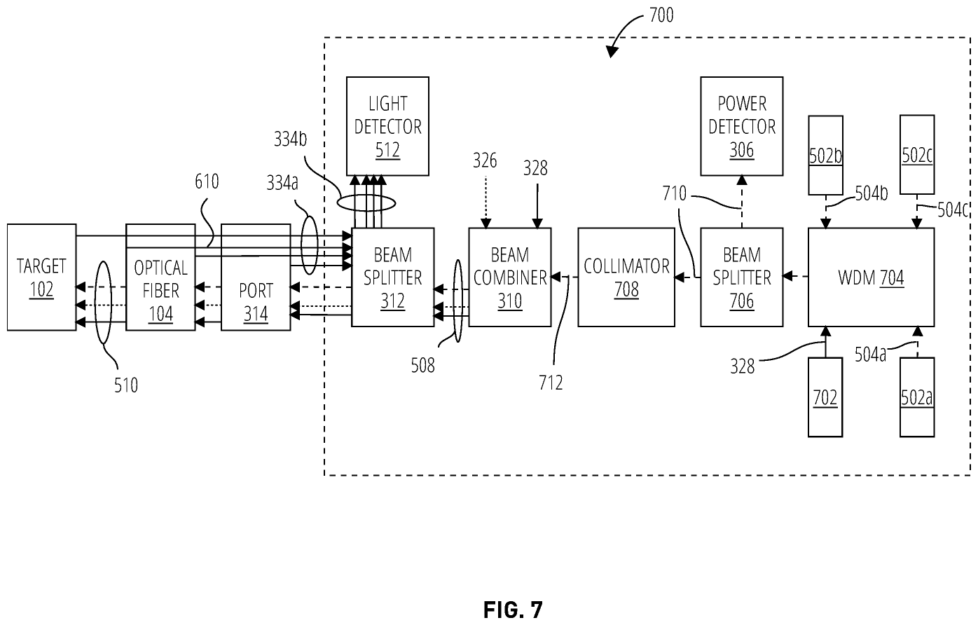

FIG. 7 shows an exemplary LETD system 700 for estimating distance between a fiber end and a target in accordance with some embodiments of the present disclosure. LETD system 700, like LETD system 500, is implemented in a non-polarized environment. Further, LETD system 700 is a “semi-fiber-based design” in which the two input beam splitters seen in the previous configurations (e.g., the configuration of LETD system 600) are replaced with a wavelength division multiplexer (WDM). The WDM power loss may be approximately 20% while the beam splitter power loss may be approximately 50%, improving efficiency of the LETD system 700 with use of the WDM. Additionally, the WDM may perfectly, or almost perfectly, align each of the three laser sources into an optical path. However, beam splitters and beam combiners are considerably less accurate at aligning each of the three laser sources into a single beam.

LETD system 700 utilizes a third non-polarized laser source 502c along with the first non-polarized laser source 502a and the second non-polarized laser source 502b. The third non-polarized laser source 502c is introduced for the purpose of calibration of the optical fiber condition in real-time as described above. It will be appreciated that the calibration laser may be polarized, or non-polarized, without departing from the scope of this disclosure. LETD system 700 may include one or more non-polarized lasers, one or more beam splitters, a beam combiner, one or more light detectors, a WDM, and a collimator. As shown in the FIG. 7, the LETD system 700 includes the non-polarized laser source 502a, the non-polarized laser source 502b, the non-polarized laser source 502c, the laser source 702, the wavelength division multiplexer (WDM) 704, the beam splitter 706, the power detector 306, the collimator 708, the beam combiner 310, the beam splitter 312 and the light detector 512. In the configuration of LETD system 700, the non-polarized laser source 502a can emit light with a wavelength having a high-water absorption coefficient (HI) while the non-polarized laser source 502b can emit light with a wavelength having a low water absorption coefficient (LO). Further, the non-polarized laser source 502c can emit light having a wavelength with a very high-water absorption coefficient (e.g., completely, or almost completely, absorbed by water) relative to the wavelength of light emitted by the laser sources 502a and 502b. As an example, the wavelength of the non-polarized laser source 502c may be 1435 nm.

As mentioned above, in the configuration of LETD system 700, the beam splitter 304 and the beam splitter 602 shown in FIG. 6 are replaced with the WDM 704. In some embodiments, to ensure correct usage of the non-polarized laser source 502c as a real-time calibrator, the incident light beams coming from each of the non-polarized lasers laser sources 502a, 502b, and 502c can be arranged to enter at the proximal end of the optical fiber 104 at the same point and at the same angle. In many embodiments, it may be difficult or impossible to align incident light beams from each of the non-polarized lasers laser sources 502a, 502b, and 502c to enter at the same point and at the same angle with combiners/splitters. To ensure adherence with this condition of same point and same angle, configuration shown in LETD system 700 utilizes WDM 704. The WDM 704 can be configured to ensures that all the incident light beams coming from each of the non-polarized lasers laser sources 502a, 502b, and 502c enter at the proximal end 202 of the optical fiber 104 at the same point and at the same angle. Moreover, in various embodiments, usage of WDM 704 may lower power loss, such as when compared to some beam splitters which cause 50%-75% power loss.

The incident light beams from laser sources 502a, 502b, and 502c as well as aiming beam 328 are provided as inputs to the WDM 704, which is configured to combine the incident light beams in a way that the light beams move identically. Further, output of the WDM 704 may be provided as an input to a fiber-based beam splitter (e.g., the fourth beam splitter 706), which can be arranged to split the incident light beams at a high transmission to reflection ratio (e.g., 95:5 or 99:1), as shown in the FIG. 7. In some embodiments, the beam splitter 706 is a fiber-based beam splitter. The power detector 306 associated with the beam splitter 706 may measure the power in the optical signal (e.g., light beam 710) corresponding to each wavelength. Further, the output of the beam splitter 706 (e.g., light beam 710), which is the incident light aligned along a single optical path, may be provided as an input to the collimator 708 to narrow down the light beams 710 into parallel beams.

Thereafter, output of the collimator 708 (e.g., light beams 712) can be provided to the beam combiner 310, which combines the light beams 712 coming out of the collimator 708 with an aiming beam 328 and the treatment beam 326 as shown in FIG. 7. In several embodiments, the aiming beam 328 may be introduced at the WDM 704. In many embodiments, the aiming beam 328 may be introduced at the beam combiner 310. Still, in some embodiments, the aiming beam 328 can be introduced at both the WDM 704 and the first beam combiner 310. In some embodiments, the aiming beam 328 and/or the treatment beam 326 may be generated by one or more laser sources other than the laser sources 502a, 502b, and 502c or the aiming beam 328 and/or the treatment beam 326 may be generated by the laser sources 502a, 502b, and 502c.

The combined light beam 508 comprising the aiming beam 328, the treatment beam 326, and the light beams 712 (e.g., e.g., light from laser sources 502a′, 502b, and 502c received from the collimator 708) may be subjected to the beam splitter 312 having a configuration of R/T ratio 50:50 and a 45-degree AOI. The beam splitter 312 may split the combined light beam 508 in the ratio of 50:50, such that, the aiming beam 328, the treatment beam 326, and the non-polarized light beams combined light beam 508 from laser sources 502a, 502b, and 502c may be aligned along a single optical path. The light beams 510, which are the output of the beam splitter 312, are then transmitted to optical fiber 104 (e.g., via port 314) as shown and more fully described above.

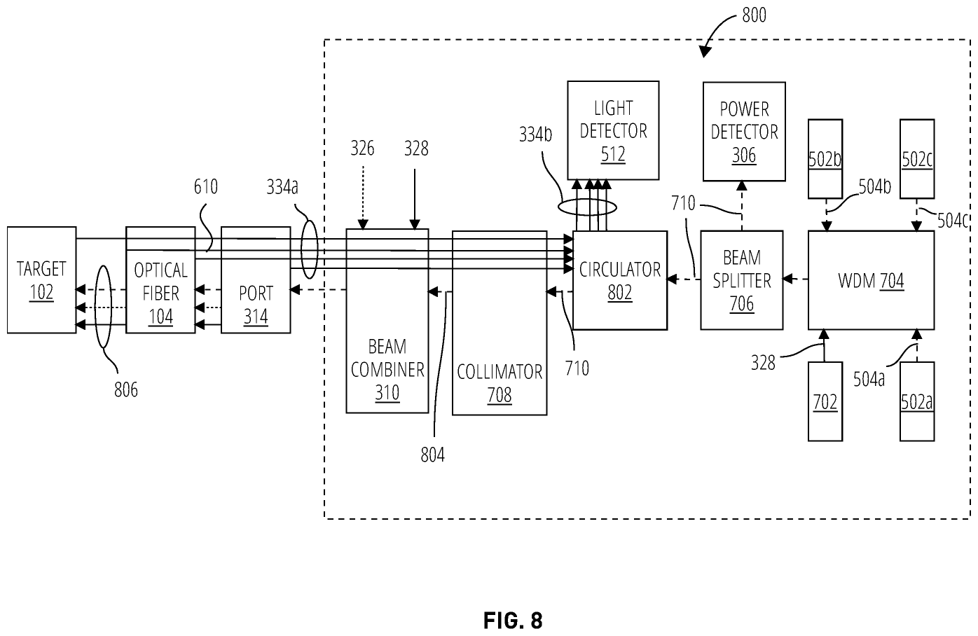

FIG. 8 shows an exemplary LETD system 800 for estimating distance between a fiber end and a target in accordance with some embodiments of the present disclosure. LETD system 800, like configurations of LETD system 500 and LETD system 700, is implemented with non-polarizing detectors. However, the sources can be non-polarized or polarized. In this exemplary configuration, LETD system 800 may include one or more non-polarized lasers (or polarized lasers), one or more beam splitters, a beam combiner, one or more light detectors, a WDM, a circulator and a collimator. As shown in the FIG. 8, LETD system 800 includes the non-polarized laser source 502a, the non-polarized laser source 502b, the non-polarized laser source 502c, the laser source 702, the wavelength division multiplexer (WDM) 704, the beam splitter 706, the power detector 306, the circulator 802, the light detector 512, the collimator 708, and the beam combiner 310. In configuration of LETD system 800, the non-polarized laser source 502a can emit light having a wavelength with a high-water absorption coefficient (HI) while the polarized laser source 502b can emit light having a wavelength with a low water absorption coefficient (LO). Further, the non-polarized laser source 502c can have a wavelength with very high-water absorption coefficient, which is substantially absorbed in water.

Like described above, in LETD system 800 the beam splitter 304 and the beam splitter 602 shown in FIG. 6 are replaced with the WDM 704, as shown in FIG. 7 and FIG. 8. Further, in LETD system 800, the beam splitter 312, which was arranged to deliver the light beams to the port 314 in all the aforementioned exemplary configurations, is also eliminated. Beam splitters reduce output power by up to 50% (or more) and reduce an additional 50% (or more) of output power upon receiving return signals. Therefore, removal of the beam splitter 312 in LETD system 800 can significantly increase the output power and the reflected signals.

The incident light beams 504a, 504b, and 504c from laser sources 502a, 502b, and 502c, respectively, as well as the aiming beam 328 are provided as inputs to the WDM 704, which is configured to combine the incident light beams in a way that the light beams move identically. Further, output of the WDM 704 may be provided as an input to the beam splitter 706 that splits the incident light beams at a ratio of 95:5. As previously mentioned, other ratios, such as 99:1, may be utilized without departing from the scope of this disclosure. In some embodiments, the beam splitter 706 is a fiber-based beam splitter, thereby rendering the configuration of LETD system 800 an all fiber-based design. The power detector 306 associated with the beam splitter 706 may measure the power in the optical signal (e.g., light beam 710) corresponding to each wavelength. Further, the output (e.g., the light beam 710) of the beam splitter 706, which is the incident light aligned along a single optical path, may be provided as an input to the circulator 802. The circulator 802 is configured to ensures that all the light beams travel in one direction. Additionally, the circulator 802 provides the light beams 804 to the collimator 708, from a port other than the port into which the light beam 710 entered. The collimator 708 may narrow down the light beams into parallel light beams 804. The circulator 802 may, when compared to beam splitters, provide (1) lower power loses (beam splitter losses are ˜50% in each direction) and (2) a more flexible optical design (free space optics require straight lines, while fiber-based designs can be folded as desired).

Output (e.g., parallel light beams 804) of the collimator 708 may be provided to the beam combiner 310, which combines the light beams 804 coming out of the collimator 708 with the aiming beam 328 and the treatment beam 326 into combined light beams 806, as shown in FIG. 8. In some embodiments, the aiming beam 328 can either be introduced at the beginning (e.g., into the WDM 704), can be introduced at the beam combiner 310, or can be introduced at both the WDM 704 and the beam combiner 310. In some embodiments, the aiming beam 328 and/or the treatment beam 326 can be generated by one or more laser sources other than the laser sources shown in this figure or the aiming beam 328 and/or the treatment beam 326 can be generated by the laser sources shown in this figure. The combined light beam light beams 806 comprising the aiming beam 328, the treatment beam 326, and the light beams 804 (e.g., light beams 504a, 504b, and 504c) received from the beam combiner 310 may be transmitted to optical fiber 104 (e.g., via a port 314), as shown in FIG. 8. The combined light beams 806 are transmitted to the proximal end 202 of the optical fiber 104, which then propagate through the length of the optical fiber 104 can be delivered to the target 102 from distal end 204 of the optical fiber 104.

As outlined above, when the light beams 806 are delivered to the target 102 via the distal end 204 of the optical fiber 104, the target 102 may reflect some portion of light away from the optical fiber 104 and some portion of the light towards the optical fiber 104, wherein the portion of light reflected towards the optical fiber 104 may re-enter the optical fiber 104, at the distal end 204. The portion of the reflected light re-entering at the distal end 204, as outlined above, is referred to as reflected light reflected light beams 334a. The reflected light beams 334a may be transmitted “backward” through the optical fiber 104 from the distal end 204 to the proximal end 202. When the reflected light beams 334a reaches the proximal end 202 of the optical fiber 104, the reflected light beams 334a may pass through the beam combiner 310 and the collimator 708 to be subjected to the circulator 802, where it is routed to the light detector 512 and measured as described above with respect to FIG. 7.

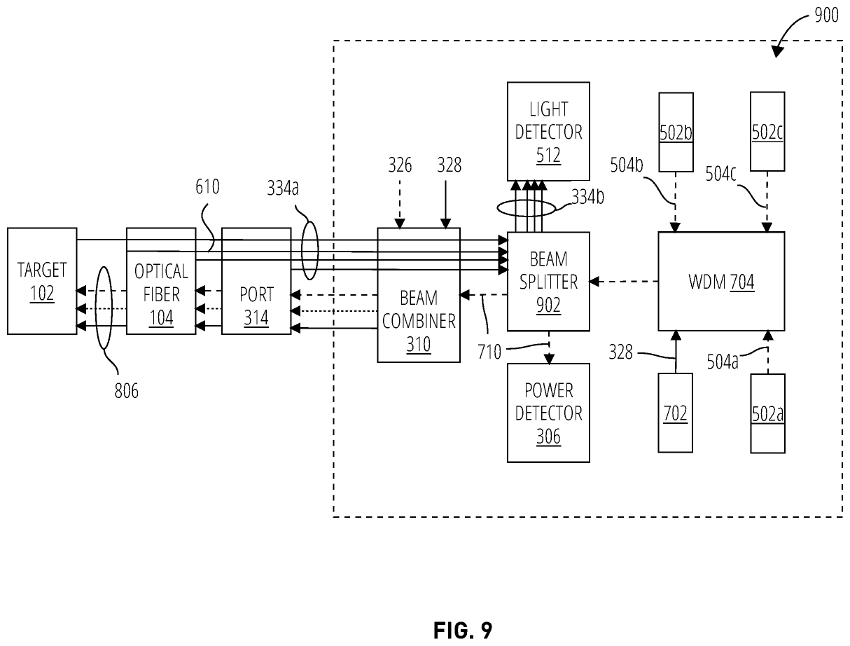

FIG. 9 illustrates LETD system 900, which like some of the prior configurations, may be implemented in a non-polarized environment. In several embodiments, LETD system 900 may include a single beam splitter based optical design. In the configuration of LETD system 900, the WDM 704 can replace the function or operation of multiple beam splitters (e.g., ones utilized in LETD system 300, LETD system 400, 500, and/or LETD system 600, or the like). The WDM 704 can receive light beams 504a, 504b, and 504c from non-polarized laser sources 502a, 502b, and 502c, respectively.

LETD system 900 may include one or more non-polarized lasers (or polarized lasers), a beam splitter, a beam combiner, one or more light detectors, a WDM, and a collimator. As shown in FIG. 9, the LETD system 900 includes the non-polarized laser source 502a, the non-polarized laser source 502b, the non-polarized laser source 502c, the laser source 702, the wavelength division multiplexer (WDM) 704, the beam splitter 902, the power detector 306, the light detector 512, and the beam combiner 310. In the LETD system 900, like in prior configurations, the non-polarized light beam 504a can have a wavelength with high water absorption coefficient (HI) relative to the non-polarized light beam 504b, which itself can have a wavelength with a lower water absorption coefficient (LO). Further, the non-polarized light beam 504c can have a wavelength with a very high-water absorption coefficient as described in detail above.

As described above, processing unit 108 can, based on readings associated with reflections of light generated by the non-polarized laser source 502c, define an optical baseline characteristic of the quality of the distal end 204 of the optical fiber 104 (e.g., the output facet, or the like). More specifically, as light from laser source 502c is highly absorbed in water, insignificant amounts of this light will be reflected back into the optical fiber 104 as part of the reflected light beams 334a. Therefore, readings associated with light reflection 610 are mainly attributable to the optical characteristics of the distal end 204 of the optical fiber 104, which as described goes through degradation during a laser treatment due to, for example, heat and cavitation. Accordingly, increased intensity readings of the light reflection 610 may indicate optical fiber tip degradation.

In several embodiments, at a certain threshold of intensity changes from the baseline reading for a specific optical fiber 103 (e.g., 10% to 50%, greater than or equal to 25%, 50%, 75%, 90%, between 10% and 100%, or the like) the processing unit 108 may indicate that the optical fiber 104 should be checked or replaced, such as through a user interface and/or audible alarm. In addition, optical fiber tip degradation may cause higher internal reflections from the distal end 204 of the optical fiber 104, of light associated with non-polarized laser sources laser sources 502a and 502b. Moreover, fiber tip degradation may change the ratios between polarities P and S in reflected light beams 334a or 334b. Therefore, creating baseline readings, for a specific optical fiber currently in use, and monitoring these baselines on the fly, may allow more accurate distance estimations even when and while the tip of the optical fiber degrades and until degradation reaches the point that an optical fiber should be replaced. Therefore, greater dynamic control of parameters associated with a therapy or treatment can be provided.

LETD system 900, like some prior configurations of LETD systems, utilizes WDM 704 to ensure that all the incident light beams coming from each of the non-polarized lasers 502a, 502b, and 502c enter at the proximal end 202 of the optical fiber 104 at the same point and at the same angle. Moreover, in various embodiments, usage of WDM 704 may lower power loss, such as when compared to some configurations utilizing beam splitters.

The light beams 504a, 504b, and 504c from the depicted laser sources as well as the aiming beam 328 can be provided as inputs to the WDM 704, which can be configured to combine the incident light beams in a way that the light beams move identically. Further, output of the WDM 704 may be provided as an input to the beam splitter 902, which can split the incident light beams at a ratio of 50:50. In some embodiments, the beam splitter 902 may be a free space (e.g., glass) based beam splitter. In some other embodiments, the beam splitter 902 may be a fiber-based beam splitter. In many embodiments, the power detector 306 associated with the beam splitter 902 may measure the power in the optical signal (e.g., light beam 710) corresponding to each wavelength.

The output of the beam splitter 902, which is the incident light aligned along a single optical path, may be provided as an input to the beam combiner 310. The beam combiner 310 may combine the light beams 710 coming out of the beam splitter 902 with the aiming beam 328 and the treatment beam 326 as shown in FIG. 9. In various embodiments, the aiming beam 328 may be introduced into the WDM 704, introduced at the beam combiner 310, or introduced at both the WDM 704 and the beam combiner 310. In several embodiments, the aiming beam 328 and/or the treatment beam 326 may be generated by one or more laser sources other than the laser sources 502a, 502b, and 502c or the aiming beam 328 and/or the treatment beam 326 can be generated by these laser sources. The combined light beams 806 comprising the aiming beam 328, the treatment beam 326, and the light beams 710 received from the beam combiner 310 can be transmitted to the optical fiber 104 (e.g., via the port 314).

As can be seen from this figure, LETD system 900 eliminates usage of multiple beam splitters. Further, since LETD system 900 utilizes a single beam splitter, it may be significantly less sensitive to treatment fiber movements and fiber bending radiuses, resulting in a more robust configuration. Moreover, since LETD system 900 has fewer optical components, such as beam splitters, beam combiners, detectors, and the like, the LETD system 900 may be more compact, simpler, and less expensive than other configurations.

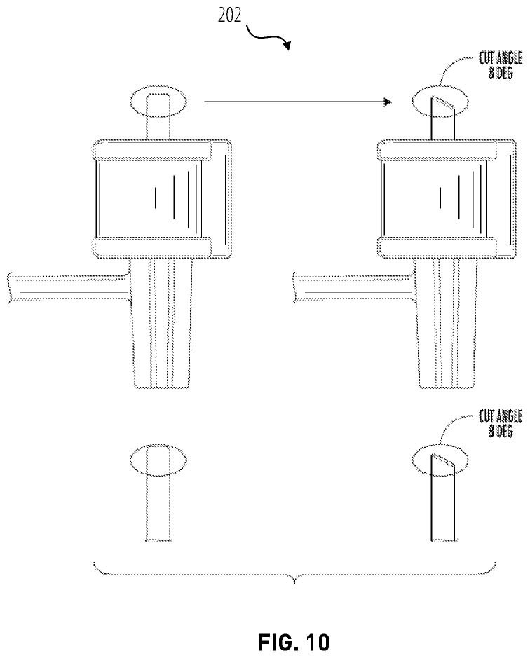

In some embodiments of the aforementioned exemplary configurations, the proximal end 202 of the optical fiber 104 can include a sub-miniature version A (SMA) connector, which may be polished or cut at an angle of 8 degrees, as shown in FIG. 10. Cutting in a slant fashion at an 8-degree angle, as shown in this figure achieves diversion of the reflected light beams (unwanted reflections caused from the proximal end 202) from the proximal end 202 of the optical fiber 104, which in turn may reduce substantive noise and increase dynamic range. In some embodiments, the light signal (e.g., reflected light beam reflected light beams 334a) that enters the light detector may contain one or more of: (a) reflections from port lens; (b) reflections from blast shield; (c) reflections from the proximal end 202 of the optical fiber 104; and/or (d) reflections from distal end 204 of the optical fiber 104.

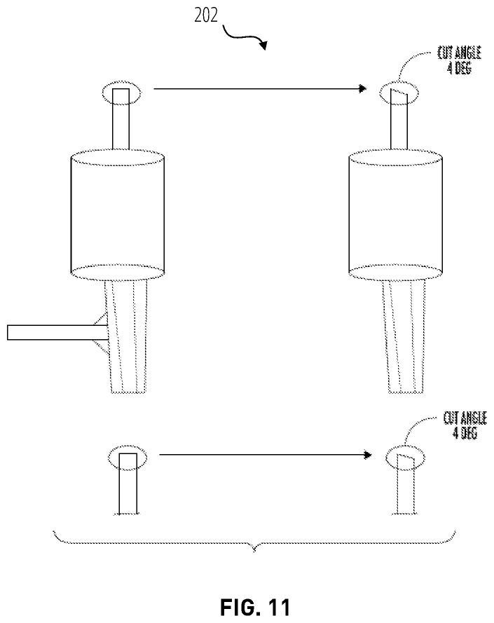

AR coating at the proximal end 202 of the optical fiber 104 may reduce reflections from the proximal end 202 of the optical fiber 104 (e.g., from 3.5% to approximately 0.5%). However, the angled finer proximal end 202 of the optical fiber 104 helps in reducing unwanted reflections and improves the dynamic range of the signals reflected from the target 102. In some other embodiments, the SMA connector can be polished or cut at an angle of 4 degrees instead of 8 degrees, for example, as shown in FIG. 11. In various embodiments, cutting in a slant fashion at a 4-degree angle, such as instead of an 8-degree (or higher) angle, may improve signal robustness. In some embodiments, the smaller the cut angles of the SMA connector may result in more signal robustness of the optical fiber 104. In various embodiments, angles from approximately 2 degrees to approximately 8 degrees may be utilized. Generally, lower angles are harder to implement in optics. In other words, it is harder to snatch it from the main signal. However, light will not enter the fiber at higher angles (e.g., 10+ degrees).

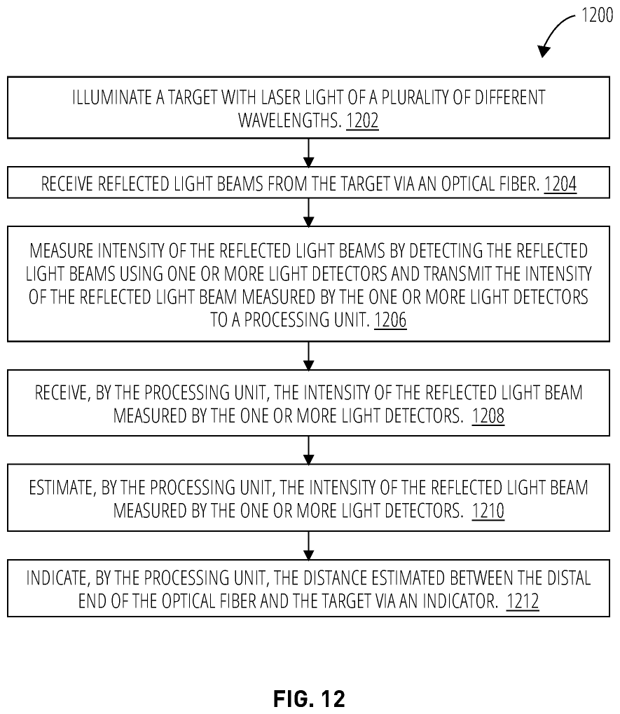

FIG. 12 illustrates a flowchart showing a method 1200 of estimating distance between a fiber end and a target in accordance with some embodiments of the present disclosure. The method 1200 is described with reference to the system 100 and to the various configurations of the LETD system 106 described above (e.g., LETD system 300, LETD system 400, LETD system 500, LETD system 600, LETD system 700, LETD system 800, LETD system 900, etc.). It is to be appreciated however, that the method 1200 could be implemented using an LETD different than that described herein. Embodiments are not limited in this context.

At block 1202, the method 1200 includes illuminating a target with laser light of a plurality of different wavelengths. For example, LETD system 106 may utilize a plurality of laser light sources (e.g., laser sources 302a and 302b, laser sources 502a, 502b, and/or 502c, etc.) to illuminate target 102 with the laser light of the plurality of different wavelengths via the optical fiber 104. In some embodiments, the laser light of the plurality of different wavelengths may be provided to the optical fiber 104 for illuminating the target 102 using one of the configurations discussed above. In various embodiments, the present disclosure may use light having two different wavelengths (e.g., light beams 320a and 320b, light beams 504a, 504b, and/or 504c, etc.) where each wavelength has a different water absorption coefficient to ensure robustness with respect to different types of targets 102, target compositions, target colors, target surfaces, and the like.

In some embodiments, the two (or more) wavelengths may be selected such that, one is a wavelength with low water absorption coefficient (LO), and another is a wavelength with high water absorption coefficient (HI). As an example, the two wavelengths may be 1310 nm and 1340 nm. However, this example should not be construed as a limitation, as different wavelengths with different water absorption coefficients can be used. For example, 1260-1320 nm may be utilized for LO and 1330-1380 nm may be utilized for HI. More generally, any combination of pairs of wavelength water absorption coefficients with a 2:1 (or greater) ratio may be utilized. In some embodiments, one or more of the following pairs may be utilized for LO and HI lasers, respectively, 1310 nm and 1340 nm lasers, 1260 nm and 1340 nm lasers, 1260 nm and 1310 nm, and 1310 nm and 1550 nm lasers. As outlined above, in some embodiments, two laser sources (e.g., 201a and 201b or 201a′ and 201b′) can be used to emit light of two different wavelengths. In some embodiments, the laser light sources can be polarized laser sources, non-polarized laser sources, or a combination of polarized and non-polarized laser sources. As an example, to measure the distance between the distal end 204 of the optical fiber 104 and the target 102, a low-power infrared (IR) laser may be used, without limitation, to illuminate the target 102 via the optical fiber 104. In other embodiments, lasers other than IR lasers may be utilized. However, IR lasers may be utilized due to it not including visible light that may disturb users.

At block 1204, the method 1200 includes receiving reflected light beams from the target via an optical fiber. For example, the LETD system 106 may receive reflected light beams 334a or reflected light beams 514a from the target 102, via the optical fiber 104. In some embodiments, the reflected light beams 334a or reflected light beams 514a may include a mixture of reflections, such as from the proximal end 202 of the optical fiber 104, from the distal end 204 of the optical fiber 104, from the port 314, from the blast shield (not shown), and the like. In various embodiments, the LETD system 106 may be configured to identify the reflected light beams suitable for measuring intensity.

At block 1206, the method 1200 includes measuring the intensity of the reflected light beams by detecting the reflected light beams using one or more light detectors and transmitting an indication (e.g., an electrical signal, or the like) of the intensity of the reflected light beam measured by the one or more light detectors to a processing unit. For example, the LETD system 106 may measure intensity of reflected light beams 334a or reflected light beams 514a, also referred to herein as returned signal, by detecting the returned signals using the one or more light detectors provided in the LETD system 106. In some embodiments, since two different wavelengths are used for illuminating the target 102, the measured intensities are with respect to two different wavelengths. Therefore, the two measured intensities corresponding to the two different wavelengths of the laser sources (e.g., laser sources 302a and 302b, or the like) may be transmitted to the processing unit 108 associated with the LETD system 106. In various embodiments, three or more different wavelengths may be utilized, measured, and/or transmitted.

At block 1208, the method 1200 includes receiving, by the processing unit, the indication of the intensity of the reflected light beams measured by the one or more light detectors. For example, processing unit 108 may receive electrical signals comprising indication(s) of the measured intensities of the reflected light beams 334a, or the like, from the LETD system 106.

At block 1210, the method 1200 includes estimating, by the processing unit, a distance between a distal end of the optical fiber and the target based on the intensity of the reflected light beams measured by the one or more light detectors. For example, processing unit 108 may estimate a distance between the distal end 204 of the optical fiber 104 and the target 102 based on the measured intensities of the returned signal. In some embodiments, the processing unit 108 may substitute the measured intensities in the Equation 1 as shown below:

Intensity of the returned signal=R*e^{(-λ*X)}: Equation 1

In the above Equation 1, “R” refers to target reflection coefficient, which is affected by target composition, target color/pigment, target angle, target surface and the like; “λ” refers to water absorption coefficient of a specific wavelength; and “X” refers to distance between the distal end 204 of the optical fiber 104 and the target 102.

In the above Equation 1, “X” and “R” are unknown parameters which need to be determined by the processing unit 108. Therefore, in order to determine the values of “X” and “R”, the processing unit 108 may substitute the two measured intensity values in the above Equation 1, thereby obtaining two equations with substituted values of measured intensity and the water absorption coefficient of the corresponding wavelength. For instance, the two equations with substituted values may be as shown below.

I_{(HI)}=R*e^{(-λ_HI*X)}: Equation 1.1

I_{(LO)}=R*e^{(-λ_LO*X)}: Equation 1.2

The processing unit 108 may further simplify the above substituted Equations 1.1 and 1.2 as follows: compute ratio of measured intensity values obtained for the returned signal of two different wavelengths using Equation 2.1; and determine distance value using the natural logarithm as shown in Equation 2.2.

$$\frac{{I}_{( HI )}}{{I}_{( LO )}} = \frac{R}{R} * {e}^{( {λ}_{LO} - {λ}_{HI} ) * X} \qquad Equation 2.1$$

$$X = \frac{ln ( \frac{{I}_{( HI )}}{{I}_{( LO )}} )}{{λ}_{LO} - {λ}_{HI}} \qquad Equation 2.2$$

Therefore, the processing unit 108 may estimate the distance X between the distal end 204 of the optical fiber 104 and the target 102, by simplifying Equations 1.1 and 1.2 as shown above. In the above Equation 2.2, “ln” refers to natural logarithm. In some embodiments, the distance X may be measured in millimeters. In some embodiments, X is the same distance for both wavelengths and R (target reflection) is almost identical for both wavelengths when the selected wavelengths are close to each other on the “nm scale”. In some embodiments, wavelengths may be considered close to each other on the “nm scale” when they are within 250 nm (e.g., 1310 nm and 1340 nm or 1310 nm and 1550 m). However, in many embodiments, wavelengths with closer R values may be selected. Accordingly, 1310 nm and 1340 nm may be selected over 1310 nm and 1550 nm. With some examples of the present disclosure, the two laser sources (e.g., laser sources 302a and 302b, or the like) can be arranged to emit light having wavelengths that are within 100 nm of each other.

The condition of the optical fiber 104 may be affected due to factors such as changes or degradation of the distal end 204 and/or proximal end 202 of the optical fiber 104, fiber bending effects on polarization scrambling, or any other degradations and changes occurring in the optical fiber 104. Changes in optical conditions of the optical fiber 104, specifically the tips/ends of the optical fiber 104, may adversely affect one or more of the quality of the irradiated beam, the intensity of the internal reflected light beams, the amount of back reflected light from a target which enters the fiber, the amount of energy that reaches a target, and the accuracy of measurements. This may affect the accuracy of the distance estimation, potentially leading to incorrect positioning of the optical fiber 104 during the treatment or miscalculating energy optimization which are based on distance estimation as described in U.S. Provisional Patent Application No. 63/118,117, which is incorporated herein by reference.

Internal reflections from planes associated with the fiber (e.g., the fiber proximal end or the fiber distal end) or planes associated with other optical elements which are optically connected with the fiber (e.g., lenses or shields) can generate parasitic and unwanted reflections. Moreover, these internal reflections may change over time due to fiber or other elements degradation. Also, fiber degradation may change the quality of the laser beam irradiated toward the target and/or the intensity of back reflected light from a target tissue, such as the reflected light that enters and passes through the optical fiber as reflected light beams 334a and 334b).

As such, with some embodiments, at block 1210, method 1200 can measure the initial internal reflections of each laser before a treatment starts to keep accurate distance measurements during fiber degradation and changes in internal reflections. In many such embodiments, the initial internal reflection values (or base values) may be recorded and utilized to monitor changes over time. For example, processing unit 108 can include circuitry (e.g., registers, memory, or the like) to store indications of the initial internal reflection values. In several embodiments, this process may be performed for one or more optical fibers 104 to be used with a laser system. For example, this process may be performed for each optical fiber 104 to be used with a laser system. Various embodiments described herein may monitor changes from the initial internal reflection values (e.g., stored in circuitry of processing unit 108, or the like) to dynamically calibrate distance measurements as provided herein.

In some embodiments, the processing unit 108 is configured to read (e.g., from a register, from memory, or the like) baseline values of such parasitic (e.g., unwanted) reflections using a system pre-treatment calibration process. In some embodiments, the system pre-treatment calibration process may include setting up a treatment fiber in water with no target. In this context, “no target” can be interpreted to mean that the closest target (e.g., a stone, a tumor, or the like) may be located far enough away from the tip of the fiber such that no light or substantially no light reflects off the target and into the optical fiber 104 as signal reflected light beams 334a. Such a distance may be, for example, 10 mm from the distal end 204 of the optical fiber 104, or more, for IR sources (e.g., 1310 nm and 1340 nm sources). However, if visual light (e.g., 400 nm-700 nm) is utilized then a length greater than 10 mm may be utilized. Thereafter, under these conditions, the system may activate the lasers (e.g., laser sources 302a and 302b, or the like) and measure the reflected signals reflected light beams 334a as described above. Since the reflected light reflected light beams 334a under these conditions (e.g., active laser in the presence of water but not target) is very low, the signals reaching the light detectors are related mainly to internal reflections associated with the optical fiber 104 (e.g., from the port 314, the proximal end 202, the distal end 204, or the like).

The internal reflected (IR) light beams in such a scenario may be detected using the light detectors and the measured intensity values may be stored as IR_{(HI)} and IR_{(LO)}, by the processing unit 108 (e.g., in a register, in memory circuitry, or the like). IR_{(HI)} may be the intensity of the internal reflections of incident light having higher water absorption coefficient when there is no target close to the tip of the optical fiber 104 (e.g., the distal end 204) while IR_{(LO)} may be the intensity of internal reflections of incident light having low water absorption coefficient when there is no target close to the optical fiber 104 (e.g., the distal end 204). Thereafter, during a therapy or treatment, when the laser is activated while the distal end 204 of the optical fiber 104 is placed at a closer distance to the target 102, reflected light beams 334a may be reflected backward through the optical fiber 104 and detected using the light detectors described herein.

In addition to calculating the measured intensity values as described above, processing unit 108, at block 1210, can store the measured intensity values (e.g., in a register, in memory circuitry, or the like) as I_{(HI)} which may be an indication of the intensity of returned signal from a target 102 (e.g., tissue, stone, etc.) corresponding to wavelengths having higher water absorption coefficient (HI) and store I_{(LO)} which may be an indication of the intensity of returned signal from a target 102 (e.g., tissue, stone, etc.) corresponding to wavelengths having lower water absorption coefficient (LO). However, to eliminate values of parasitic (or unwanted) reflections from readings of the actual reflected light beams 334a, the processing unit 108 may subtract and/or reduce the IR_{(HI)} from reading of the actual returned signal I_{(HI)} as shown in the below Equation 3.1, and IR_{(LO)} from reading of the actual returned signal I_{(LO)} as shown in the below Equations 3.1 and 3.2, respectively.

I′_{(HI)}=I_{(HI)}−IR_{(HI)}: Equation 3.1

I′_{(LO)}=I_{(LO)}−IR_{(LO)}: Equation 3.2

In the above Equation 3.1, I′_{(HI)} refers to a new calculated intensity of returned signals corresponding to wavelengths having higher water absorption coefficient (HI) (without the parasitic (or unwanted) reflections); I_{(HI)} refers to a measured intensity of returned signal corresponding to wavelengths having higher water absorption coefficient (HI) (with the parasitic (or unwanted reflections); and IR_{(HI)} refers to a measured intensity of internal reflections of incident light having higher water absorption coefficient (measured with “no target”).

Similarly, in the above Equation 3.2, I′_{(LO)} refers to a new calculated intensity of returned signals corresponding to wavelengths having lower water absorption coefficient (LO) (without the parasitic (or unwanted) reflections); I_{(LO)} refers to a measured intensity of returned signals corresponding to wavelengths having lower water absorption coefficient (LO) (with the parasitic (or unwanted) reflections); and IR_{(LO)} refers to measured intensity of internal reflections of incident light having lower water absorption coefficient (measured with “no target”).

Therefore, using the new intensity calculated values I′_{(HI)} and I′_{(LO)}, the processing unit 107 may determine the distance between the distal end 113 of the optical fiber 103 and the target 101, by substituting the new “calibrated” values I′_{(HI)} and I′_{(LO)}, into Equation 2.2 as shown below:

$$X = \frac{ln ( \frac{{I}_{( HI )} - {IR}_{( HI )}}{{I}_{( LO )} - {IR}_{( LO )}} )}{{λ}_{LO} - {λ}_{HI}}$$

In some embodiments, the above equation of “X” may also be indicated as shown below:

$$X = \frac{ln ( \frac{{I}_{( HI )}^{′}}{{I}_{( LO )}^{′}} )}{{λ}_{LO} - {λ}_{HI}}$$

As mentioned above, the internal reflections may not be constant over time and may change due to some changes in internal optical parameters of the system (as opposed to changes due to the dynamics of the treatment environment which is external to the system) such as the optical quality of the distal end 204 of the optical fiber 104. Due to one or more of the power level of the treatment beam 326, cavitation effects that take place at the distal end 204 (or tip) of the optical fiber 104, and the liquid environment in which the fiber is disposed during treatment, the optical fiber undergoes various amounts of degradation, primarily at the distal end 204 (or the tip). Accordingly, in several embodiments, “real-time” or “dynamic” calibration may be performed by monitoring the reflected light beams 334a repeatedly during a treatment and dynamically accounting for or adjusting for such changes in internal reflections. For example, for performing such real-time calibration, as shown in the configurations described herein using a calibration laser (e.g., laser source 302c, or the like) can be utilized to facilitate more accurate distance estimation that accounts for such degradation of the optical fiber 104.

As explained with respect to these configurations, the calibration laser beam (e.g., light beams 320c, or the like') has a wavelength with a very high absorption coefficient in water. As an example, the wavelength of the polarized laser source 302c, or non-polarized laser source 502c may be 1435 nm. Since laser beams generated by the these “calibration” laser sources are so strongly absorbed by the liquid environment, as explained above, hardly any reflected light beams 334a associated with these laser beams goes back into the fiber. Therefore, while the calibration laser source is active, the reflected light beams 334a having a wavelength of the calibration laser source mainly are associated with (or indicative of) internal reflections.

In several embodiments, processing unit 108, at block 1210, can be configured to read and store one or more base values for the internal reflections of the system 100 associated with the laser sources laser source 302c (or etc.) before a treatment starts. These one or more base values may represent the “quality” of the optical fiber 104 (e.g., the optical quality of the distal end 204) before the treatment starts and can be stored (e.g., in a register, in memory circuitry, or the like) by the processing unit 108. Further, processing unit 108 may be configured to continue measuring, in “real-time” (e.g., periodically, repeatedly, or the like) during a treatment, internal reflections of light emitted by the calibration laser source (e.g., laser source 302c, etc.) to identify deviations from the base values. Monitoring these deviations provides an indication as to a degradation of the optical quality of the optical fiber 104 and may be used to correct any measured back reflected intensity associated with reflected light beams 334a. In many embodiments, based on the readings of the internal reflections of light emitted by the calibration laser sources, processing unit 108 may rectify calibration parameters for the main laser sources (e.g., laser sources 302a and 302b, etc.).

In some embodiments, method 1200 can include a block for a calibration process. For example, processing unit 108 can read and store one or more internal reflections values associated with light emitted by the calibration laser sources where the system 100 is activated in water. Since calibration laser sources are so highly absorbed in water, there may be much less sensitivity, relative to measurements of reflected signals associated with light emitted by the other laser sources, to the distance to a target 102 during the calibration readings of the calibration laser sources. As will be explained in more detail below, this can provide the continuation of calibration laser measurements during treatment when a target may also be close to the tip of the fiber.

Thereafter, the target 102 can be illuminated using one of the exemplary configurations with the non-calibration laser sources. The reflected light beams 334a and reflected light beams 334b in such a scenario may be detected using the light detectors and the processing unit 108 can store the measured intensity values as I_{(HI)}, I_{(LO)} together with additional and associated measurements of the internal reflections of calibration laser IR_{(CAL)}. I_{(HI)} may be the intensity of the back reflections from the target of incident light having a higher water absorption coefficient, I_{(LO)} may be the intensity of the back reflections from the target of incident light having a low water absorption coefficient, and IR_{(CAL)} may be the intensity of the internal reflections of incident light from calibration laser sources.

In some embodiments, the presence or absence of a target 102 may not affect the reflections IR_{(CAL)} because the incident light from the calibration laser sources are highly absorbed by water. As a result, changes in the IR_{(CAL)} value may be a result of changes in degradation of the optical fiber 104, specifically the tips (e.g., the distal end 204, or the like) of the optical fiber 104. In some embodiments, based on relative changes of the IR_{(CAL)} value, the processing unit 108 may adjust the previously measured IR_{(HI)} and IR_{(LO)} values or the currently measured I_{(LO)} or I_{(HI)}.

Thereafter, during a treatment (e.g., when the laser is activated to treat a target 102) when there is the presence of the target 102 (e.g., when the target 102 is at a distance close enough to generate reflected light beams 334a, such as when the target 102 is in a distance less than or equal to 10 mm from the distal end 204 of the optical fiber 104), the reflected light beams 334a for laser sources 302a or 502a and for laser sources 502a or 502b and the light reflection 610 from the calibration laser sources 302c or 502c may be detected using the light detectors. The processing unit 108, at block 1210, can store the measured intensity values as I_{(HI)} which may be representative of the intensity of returned signals corresponding to light having wavelengths with a higher water absorption coefficient (HI), I_{(LO)} which may be representative of the intensity of returned signals corresponding to light having wavelengths with a lower water absorption coefficient (LO), and IR_{(CAL)} which may be representative of the intensity of returned internal reflection signals corresponding to light having wavelengths with a higher still water absorption coefficient (e.g., light emitted by the calibration laser sources 302c or 502c). Further, to determine a calibration factor, the processing unit 108 may divide IR_{(CAL-PRE)} from the calibration process pre-treatment from IR_{(CAL-DUR)} from a calibration process done during a treatment as shown in the below Equation 4.

$$Calibration Factor ( CF ) = \frac{{IR}_{( CAL - PRE )}}{{IR}_{( CAL - DUR )}} \qquad Equation 4$$

When the internal reflections of the calibration laser sources before and during a treatment are the same and there are no changes in the optical fiber 104 the calibration factor may be “1”. Further, to rectify parameters for the main laser sources 302a and 302b or laser sources 502a and 502b based on the calibration factor, the processing unit 108 may use the calibration factor as shown in the below Equations 5.1 and 5.2.

I″_{(HI)}=I_{(HI)}−IR_{(HI)}×CF: Equation 5.1

I″_{(LO)}=I_{(LO)}−IR_{(LO)}×CF: Equation 5.2

In the above Equation 5.1, I″_{(HI)} refers to a new calibrated intensity of back reflected signals from the target 102, which corresponds to light having wavelengths with a higher water absorption coefficient (HI); I_{(HI)} refers to the measured intensity of the back reflected signals from the target 102 which is corresponding to light having wavelengths with the higher water absorption coefficient (HI); IR_{(HI)} refers to the measured intensity of the internal reflection of incident laser light having wavelengths with the higher water absorption coefficient (measured with “no target”); and CF refers to calibration factor determined using Equation 4.

In the above Equation 5.2, I″_{(LO)} refers to a new calibrated intensity of back reflected signals from a target which is corresponding to light having wavelengths with a lower water absorption coefficient (LO); I_{(LO)} refers to the measured intensity of back reflected signals from a target which is corresponding to light having wavelengths with the lower water absorption coefficient (LO); IR_{(LO)} refers to the measured intensity of internal reflection of incident laser light having wavelengths with the lower water absorption coefficient (measured with “no target”); and CF refers to calibration factor determined using Equation 4.

Therefore, using the new calibrated intensity values I″_{(HI)} and I″_{(LO)}, the processing unit 108 at block 1210, can determine the distance between distal end 204 of the optical fiber 104 and the target 102, by substituting the new calibrated values I″_{(HI)} and I″_{(LO)}, into Equation 2.2 as shown below:

$$X = \frac{ln ( \frac{{I}_{( HI )} - {IR}_{( HI )} * CF}{{I}_{( LO )} - {IR}_{( LO )} * CF} )}{{λ}_{LO} - {λ}_{HI}}$$

Therefore, in this way, system pre-treatment calibration and real-time calibration may be performed and utilized to update the calibration factor (e.g., via processing unit 108) in real-time to dynamically account for changes (e.g., degradation, or the like) of the optical fiber 104 during operation. In several embodiments the pre-treatment and real-time calibrations may be performed to ensure the accuracy of the estimated distance between the distal end 204 of the optical fiber 104 and the target 102 when the optical fiber 104 undergoes degradation.

At block 1212, the method 1200 includes indicating, by the processing unit 108, the distance estimated (e.g., at block 1210) between the distal end 204 of the optical fiber 104 and the target 102 via an indicator. For example, the processing unit 108 cause the estimated distance between the distal end 204 of the optical fiber 104 and the target 102 to be indicated via an indicator 110 associated with the processing unit 108. As a specific example, the indicator 110 may include one or more of a visual indicator, an audio indicator, and a haptic indicator. Accordingly, processing unit 108, at block 1210, can send a control signal to the indicator 110 to cause the indicator to indicate (e.g., display, audibly signal, haptically signal, or the like) an indication of the estimated distance.

In some embodiments, based on the estimated distance between the distal end 204 of the optical fiber 104 and the target 102, one or more of the position of the optical fiber 104, the orientation of the optical fiber 104, characteristics of the treatment beam, and the like may be varied, in real-time, to affect the treatment beam accurately and efficiently on the target 102, such as through more accurate aiming.

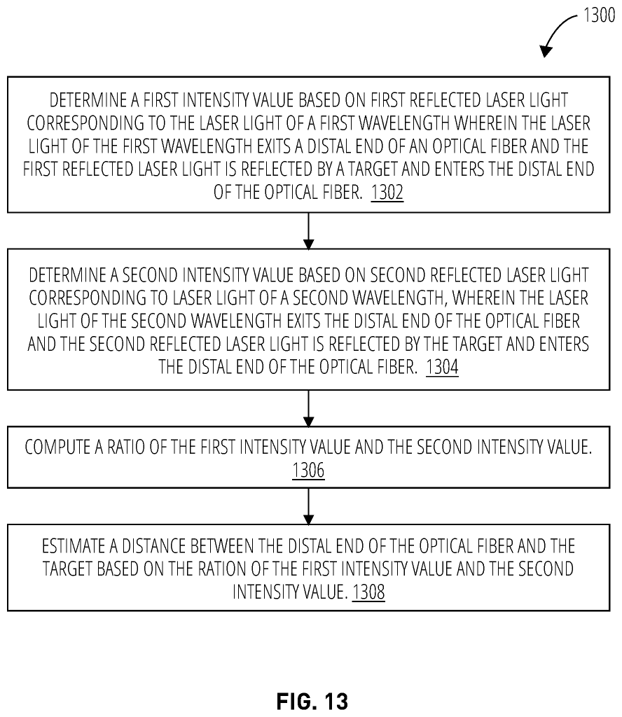

FIG. 13 illustrates a flowchart showing a method 1300 of estimating a distance between a fiber end and a target in accordance with some embodiments of the present disclosure. The method 1300 is described with reference to the system 100 and to the various configurations of the LETD system 106 described above. It is to be appreciated however, that the method 1300 could be implemented using a different LETD system than that described herein. Embodiments are not limited in this context.

At block 1302, the method 1300 includes determining a first intensity value based on first reflected laser light corresponding to laser light of a first wavelength, wherein the laser light of the first wavelength exits a distal end 204 of an optical fiber 104, and the first reflected laser light is reflected by a target 102 and enters the distal end 204 of the optical fiber 104. For example, processing unit 108 may determine a first intensity value based on reflected light beams 334a corresponding to light having a wavelength with a high-water absorption coefficient. In some embodiments, the laser light corresponding to the wavelength having a high-water absorption coefficient may be generated by laser sources 302a or 502a, as discussed above.

At block 1304, the method 1300 includes determining a second intensity value based on a second reflected laser light corresponding to laser light of a second wavelength, wherein the laser light of the second wavelength exits the distal end 204 of an optical fiber 104 and the second reflected laser light is reflected by the target 102 and enters the distal end 204 of the optical fiber 104. For example, processing unit 108, at block 1304, may determine a second intensity value based on reflected light beams 334a corresponding to light having a wavelength with a low water absorption coefficient. In some embodiments, the laser light corresponding to the wavelength having a low water absorption coefficient may be generated by laser sources 302b or 502b, as discussed above.

At block 1306, the method 1300 includes computing a ratio of the first intensity value and the second intensity value. For example, processing unit 108 at block 1306, may utilize Equation 2.1 to compute the ratio of the first intensity value and the second intensity value. At block 1308, the method 1300 includes estimating a distance between the distal end 204 of the optical fiber 104 and the target 102 based on the ratio of the first intensity value and the second intensity value derived at block 1306. For example, processing unit 108, at block 1308, may utilize Equation 2.2 to estimate the distance between the distal end 204 of the optical fiber 104 and the target 102 based on the ratio of the first intensity value and the second intensity value.

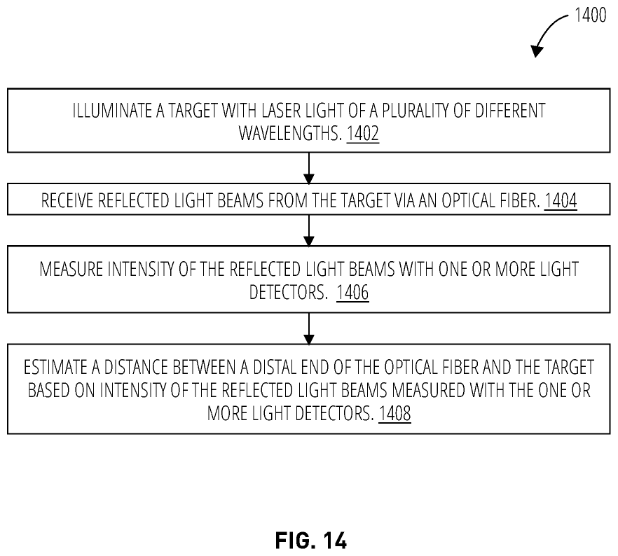

FIG. 14 illustrates a flowchart showing a method 1400 of estimating distance between a fiber end and a target in accordance with some embodiments of the present disclosure. The method 1400 is described with reference to the system 100 and to the various configurations of the LETD system 106 described above. It is to be appreciated however, that the method 1400 could be implemented using an LETD system different than that described herein. Embodiments are not limited in this context.

At block 1402, the method 1400 includes illuminating a target with laser light of a plurality of different wavelengths. For example, one of the configurations described above may be utilized to illuminate target 102 with light beams 332 having a plurality of different wavelengths. For example, the light beams 332 can include light beams 320a, 320b, 320c, treatment beam 326, and/or 328 (or the like).

At block 1404, the method 1400 includes receiving reflected light beams from the target via an optical fiber. For example, one of the configurations discussed herein can be utilized to receive reflected light beams 334a (e.g., corresponding to light reflected from the target 102) and back transmitted via optical fiber 104. In several embodiments, the reflected light beams 334a can be reflected off the target 102 and enter the distal end 204 of the optical fiber 104 and as such may include reflected light beams 334a. The reflected light beams 334a can also include light reflected from optical components within the system (e.g., the proximal end 202, the distal end 204, or the like) and can include light reflection 610 which corresponds to reflected light associated with calibration light beam 320c.

At block 1406, the method 1400 includes measuring intensity of the reflected light beams 334a with one or more light detectors. In many embodiments, one of the configurations discussed herein can be utilized to measure the intensity of the reflected light beams 334a with one or more light detectors. For example, light detectors light detector 318a and 318b may be utilized to measure the intensity of the reflected light beams 334b. In another example, other light detectors (e.g., light detector 512, or the like) may be utilized to measure the intensity of the reflected light beams 334a.

At block 1408, the method 1400 includes estimating a distance between a distal end 204 of the optical fiber 104 and the target 102 based on intensity of the reflected light beams 334a measured with the one or more light detectors. For example, processing unit 108 may be utilized to estimate the distance between a distal end 204 of the optical fiber 104 and the target 102 based on intensity of the reflected light beams 334a measured with the one or more light detectors.

It is to be appreciated that the optical elements of the LETD systems described herein (e.g., beam splitters, polarizers, beam combiners, collimator, circulator, WDM, etc.) are not constant throughout time. That is, their optical properties may change during a procedure, for example, due to thermal and environmental changes, mechanical vibrations, exposure to the treatment laser beam (e.g., high power laser beam), or other reasons. As the optical components change, the system (e.g., processing unit 108, or the like) is unable to properly differentiate between a change in the distance between the distal end 204 of the optical fiber 104 and the target 102 or a change in the optical components. As such, the present disclosure provides to capture or “snatch” the light reflected from the proximal end 202 of the optical fiber 104 to use as a reference or calibration light for the optical components such that the distance between the distal end 204 of the optical fiber 104 and the target 102 can be accurately determined even when the optical components change over time.

It is to be appreciated that the reflected light beams 334a have an incidence angle different than zero, such that the light reflected off the proximal end 202 of the optical fiber 104 has a different light path than the main treatment laser path. In general, the present disclosure provides LETD system configurations with a mirror to direct light off the optical path and into a polarized beam splitter with two detectors. The analysis of these detectors' signals gives the measure of the system changes compared to the initial state.

In some embodiments, in each of the exemplary configurations described herein, the proximal end of optical fiber 104 may be coated with a special coating such as an anti-reflective (AR) coating. The AR coating can help in reducing noise created at the proximal end 202 of the optical fiber 104 and increase the dynamic range. In some embodiments, the light signal that enter the light detector may contain one or more of: (a) reflections from a port lens; (b) reflections from a blast shield; (c) reflections from the proximal end 202 of the optical fiber 104; and/or (d) reflections from the distal end 204 of the optical fiber 104.

In various embodiments, an AR coating for the blast shield may reduce reflections from the port lens to less than 1%, an AR coating for the port lens may reduce reflections from the blast shield to less than 1%, and an AR coating at the proximal end 202 of the optical fiber 104 may reduce reflections from the proximal end 202 of the optical fiber 104 from 3.5% down to approximately 0.5%. In some embodiments, the reflected signal from a target 102 such as stone, may be of very low energy, for instance nearly 1% of fiber output power where the distance from the optical fiber tip to the tissue is about 0 mm. By reducing the reflections from the proximal end 202 of the optical fiber 104 to nearly 0.5%, the present disclosure may help in improving the dynamic range of the signals reflected from the target 102.

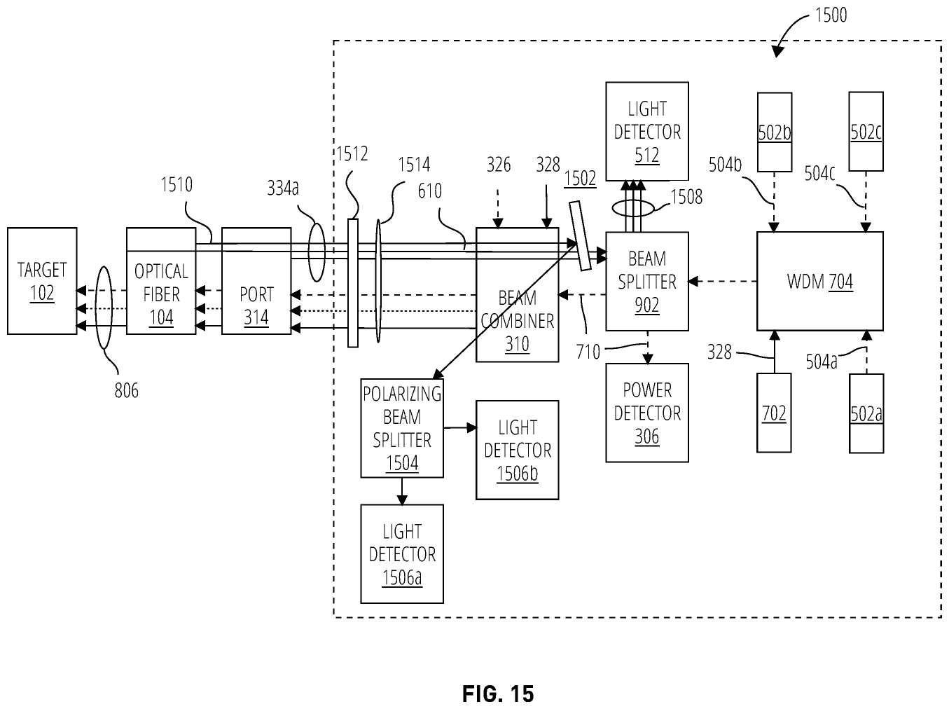

FIG. 15 illustrates a LETD system 1500, which is like the LETD system 900 but with additional “light snatch” components described herein. For example, LETD system 1500 includes the laser sources 502a, 502b, 502c, and laser source 702 as well as the WDM 704, the beam splitter 902, the light detector 512, the power detector 306, and the beam combiner 310. LETD system 1500 further includes mirror 1502, polarizing beam splitter 1504, and light detector 1506a. During operation, mirror 1502 can direct light reflection from the optical path of reflected light beams 334a towards polarizing beam splitter 1504. Said differently, mirror 1502 is provided to redirect light reflection 1510 off optical path of reflected light beams 334a such that 1510 does not reach the original optical detectors (e.g., light detector 512). It is noted that this figure does not depict the angles of incidence of the various light beams. However, it is to be appreciated that angles of the light beams are not often incident on the various optical components at 90 degrees as shown. For example, the light reflection 1510 may have an angle of incidence of 4 degrees (or the like). Further, although the mirror 1502 is depicted in the optical path of light reflection 1510 between the beam combiner 310 and beam splitter 902, the mirror 1502 could instead be disposed in the optical path between the port 314 and the 310. Further, with some examples, the mirror 1502 can be disposed and arranged such that is does not block the light beams 806 but merely redirects the light reflection 1510 as depicted and described.

As will be appreciated, as the light reflection 1510 is linearly polarized, it maintains its polarization after being reflected off the proximal end 202 of the optical fiber 104. Light reflection 1510 is directed via mirror 1502 to polarizing beam splitter 1504, where it is split into two component light beams having a polarization parallel to the light incident on the proximal end 202 of the optical fiber 104 and a polarization perpendicular to the light incident on the proximal end 202 of the optical fiber 104.

LETD system 1500 further includes the light detectors 1506a and 1506b arranged to measure an intensity of the light reflection 1510 split by the polarizing beam splitter 1504. In particular, the light detectors 1506a and 1506b are arranged to measure the intensity of the light based on polarization as outlined herein. However, of note, the parallel component of the light reflection 1510 will include both the light reflection 1510 and some portion of light reflection 610 while the perpendicular component of the light reflection 1510 will only include some portion on the light reflection 610.

With some embodiments, the distance between the distal end 204 of the optical fiber 104 and the target 102 can be represented by Equation 6.1 shown below.

$$X = \frac{ln ( \frac{{I}_{HI}}{{I}_{L O}} )}{2 ( {λ}_{L O} - {λ}_{HI} )} \qquad Equation 6.1$$

Accounting for the fact that each laser source is fired with a different illumination pulse and energy, the intensities can be normalized using the Equation 6.2 where I_{refHI} is the intensity of the reference power detector 306 for the high-water absorption coefficient light source, I_{refLO} is the intensity of the reference power detector 306 for the low water absorption coefficient light source, and I_{normHI} and I_{normLo} are the normalized intensities.

$$X = \frac{ln ( \frac{{I}_{HI} / {I}_{{ref}_{HI}}}{{I}_{LO} / {I}_{{ref}_{LO}}} )}{2 ( {λ}_{L O} - {λ}_{H I} )} = \frac{ln ( \frac{{I}_{{norm}_{HI}}}{{I}_{{norm}_{LO}}} )}{2 ( {λ}_{L O} - {λ}_{H I} )} \qquad Equation 6.2$$

The “pure” reflected signal can be represented by Equation 6.3 where I_{DETλ} is the reading from the detector for wavelength λ, I_{distλ} is the reading of the reflection from the distal end 204 of the optical fiber 104 for wavelength λ, I_{lensλ} is the reading of the reflection from the focus lens 1514 of the optical fiber 104 for wavelength λ, and I_{sigλ} is the signal to isolated (e.g., the “snatched” light signal as described herein) for wavelength λ. It is noted, that Equation 6.3 assumes that the reflections from the proximal end of the fiber are eliminated in the optical path (e.g., reduced to zero) and that reflections from the blast shield 1512 are also eliminated.

I_{DETλ}=I_{sigλ}+I_{distλ}+I_{lensλ}: Equation 6.3

LETD system 1500 can be arranged to calibrate the optical system by deriving: Iₗₑₙₛ₋₀_{_{λ}}, which is the reflectance calibration of the wavelength λ, from the focus lens 1514; I_{DET-∞}_{_{λ}}, which is the detector reading of ∞ in wavelength λ and is the sum of I_{dist-∞}_{_{λ}} or the reflection from the distal end 204 of the optical fiber 104 at ∞ in wavelength λ and the Iₗₑₙₛ₋₀_{_{λ}}, which is defined above. It is to be appreciated that the calibration is not a function or time (or rather, is constant over a procedure).

Isolating the signal from the detector in Equation 6.3 above results in I_{sig}_{_{λ}}=I_{DET}_{_{λ}}−(I_{dist}_{_{λ}}+Iₗₑₙₛ_{_{λ}}). Taking I_{dist}_{_{λ}} to be a function of time, this value can be calculated using a particular wavelength. Using 1435 nm as a specific example results in Equation 6.4 below, which is the correction of the distal tip reflection.

$${I}_{dis {t}_{λ}} = {I}_{dist - {∞}_{λ}} * ( \frac{{I}_{dis {t}_{cal}}}{{I}_{dist - {∞}_{cal}}} ) \qquad Equation 6.4$$

During operation, signals are taken from the detector, which results in Equation 6.5, which is the correction of the distal tip reflection from the actual detector.

$${I}_{dis {t}_{λ}} = ( {I}_{D E T - {∞}_{λ}} - {I}_{lens - {0}_{λ}} ) * ( \frac{{I}_{D E {T}_{cal}} - {I}_{lens - {0}_{cal}}}{{I}_{D E T - {∞}_{cal}} - {I}_{l e n s - {0}_{cal}}} ) \qquad Equation 6.5$$

Substituting the above into Equation 6.5 results in Equation 6.6, which is the signal value extracted from the signal detector (e.g., the constant static reflections).

$${I}_{{sig}_{λ}} = {I}_{D E {T}_{λ}} - \{ {I}_{lens - {0}_{λ}} + ( {I}_{DET - {∞}_{λ}} - {I}_{lens - {0}_{λ}} ) * [ \frac{( {I}_{dis {t}_{cal}} - {I}_{lens - {0}_{cal}} )}{( {I}_{dist - {∞}_{cal}} - {I}_{lens - {0}_{cal}} )} ] \} \qquad Equation 6.6$$

As will be appreciated, the addition of transmittance will change the optical path. In particular, several optical components may experience changes in their transmittance and/or reflection values. For example, the focus lens (transmittance referenced as Trₗₑₙₛ_{_{λ}}), the blast shield (transmittance referenced as Tr_{blast}_{_{λ}}), and the optical fiber 104 (transmittance referenced as Tr_{fiber}_{_{λ}}) may experience changes over time. That is, the signals that are susceptible to these transmittances are the I_{sig}_{_{λ}} and the I_{sig}_{_{λ}}. Accordingly, a final Equation for the signal with non-static reflections can be defined using Equation 6.7.

$${I}_{{sig}_{λ}} = \{ {I}_{{DET}_{λ}} - \{ {I}_{{lens}_{λ}} + ( {I}_{DET - {∞}_{λ}} - {I}_{lens - {0}_{λ}} ) * [ \text{} \frac{( {I}_{{DET}_{cal}} * {Tr}_{{lens}_{cal}}^{2} * {Tr}_{{blast}_{cal}}^{2} * {Tr}_{{fiber}_{cal}}^{2} - {I}_{lens - {0}_{cal}} )}{\begin{matrix}( {I}_{DET - {∞}_{cal}} * {Tr}_{lens - {∞}_{cal}}^{2} * \\ {Tr}_{blast - {∞}_{cal}}^{2} * {Tr}_{fiber - {∞}_{cal}}^{2} - {I}_{lens - {0}_{cal}} )\end{matrix}} ] \} \} * \frac{1}{{Tr}_{{lens}_{λ}}^{2} * {Tr}_{{blast}_{λ}}^{2} * {Tr}_{{fiber}_{λ}}^{2}} \qquad Equation 6.7$$

It is noted that the detector values I_{DET}_{_{cal}}*Trₗₑₙₛ_{_{cal}}²*Tr_{blast}_{_{cal}}²*Tr_{fiber}_{_{cal}}² are measured as a whole, without the option to differentiate the different factors from the detector. However, Equation 6.8 shown below details transmittance as a function where the lens, blast shield, and fiber are constants and can be considered, which can be like the normalized signals described above.

$${I}_{{sig}_{λ}} = {I}_{{DET}_{λ} -} \{ {I}_{lens - {0}_{λ}} + ( {I}_{D E T - {∞}_{λ}} - {I}_{lens - {0}_{λ}} ) * [ \frac{( {I}_{d i s {t}_{cal}} - {I}_{l e n s - {0}_{cal}} )}{( {I}_{d i s t - {∞}_{cal}} - {I}_{l e n s - {0}_{cal}} )} ] \} , which equals$$

$${I}_{D E {T}_{λ}} - \{ {I}_{D E T - {∞}_{λ}} \cdot [ \frac{( {I}_{dis {t}_{cal}} - {I}_{lens - {0}_{cal}} )}{( {I}_{dist - {∞}_{cal}} - {I}_{lens - {0}_{c a l}} )} ] + {I}_{l e n s - {0}_{λ}} \cdot [ 1 - \frac{( {I}_{dis {t}_{cal}} - {I}_{lens - {0}_{cal}} )}{( {I}_{dist - {∞}_{cal}} - {I}_{l e n s - {0}_{cal}} )} ] \} \qquad Equation 6.8$$

However, by applying the correction over the lens along with the distal end 204 without the lens reflection separately Equation 6.6 can be reduced as shown in Equation 6.9 below.

$${I}_{s i {g}_{λ}} = {I}_{D E {T}_{λ}} - \{ {I}_{D E T - {∞}_{λ}} * [ \frac{( {I}_{dis {t}_{c a l}} - {I}_{lens - {0}_{cal}} )}{( {I}_{dist - {∞}_{cal}} - {I}_{lens - {0}_{cal}} )} ] \} \qquad Equation 6.9$$

It will be appreciated that the component of the lens reflectivity

$$( e . g . , {I}_{l e n s - {0}_{λ}} * [ 1 - \frac{( {I}_{dis {t}_{cal}} - {I}_{lens - {0}_{cal}} )}{( {I}_{dist - {∞}_{cal}} - {I}_{lens - {0}_{cal}} )} ] )$$

is missing from Equation 6.9. Further reducing the equation by neglecting the existence of the lens entirely results in Equation 6.10.

$${I}_{s i {g}_{λ}} = {I}_{D E {T}_{λ}} - \{ {I}_{D E T - {∞}_{λ}} * [ \frac{( {I}_{dis {t}_{cal}} )}{( {I}_{dist - {∞}_{c a l}} )} ] \} \qquad Equation 6.1$$

Combining the above equations, we can define the distance as a function of luminance and reflectivity as shown in Equation 6.11.

$$X = \frac{ln ( \frac{\frac{\begin{matrix}\{ {I}_{{DET}_{high}} - \{ {I}_{{lens}_{high}} + ( {I}_{DET - {∞}_{high}} - {I}_{lens - {0}_{high}} ) \cdot \\ [ \frac{( {I}_{{DET}_{cal}} \cdot {Tr}_{{lens}_{cal}}^{2} \cdot {Tr}_{{blast}_{cal}}^{2} \cdot {Tr}_{{fiber}_{cal}}^{2} - {I}_{lens - {0}_{cal}} )}{( \begin{matrix}( {I}_{DET - {∞}_{cal}} \cdot {Tr}_{lens - {∞}_{cal}}^{2} \cdot \\ {Tr}_{blast - {∞}_{cal}}^{2} \cdot {Tr}_{fiber - {∞}_{cal}}^{2} - {I}_{lens - {0}_{cal}} )\end{matrix} )} ] \} \} \cdot \\ \frac{1}{{Tr}_{{lens}_{high}}^{2} \cdot {Tr}_{{blast}_{high}}^{2} \cdot {Tr}_{{fiber}_{high}}^{2}}\end{matrix}}{{I}_{DET - {ref}_{high}}}}{\frac{\begin{matrix}\{ {I}_{{DET}_{low}} - \{ {I}_{{lens}_{low}} + ( {I}_{DET - {∞}_{low}} - {I}_{lens - {0}_{low}} ) \cdot \\ [ \frac{( {I}_{{DET}_{cal}} \cdot {Tr}_{{lens}_{cal}}^{2} \cdot {Tr}_{{blast}_{cal}}^{2} \cdot {Tr}_{{fiber}_{cal}}^{2} - {I}_{lens - {0}_{cal}} )}{( \begin{matrix}( {I}_{DET - {∞}_{cal}} \cdot {Tr}_{lens - {∞}_{cal}}^{2} \cdot \\ {Tr}_{blast - {∞}_{cal}}^{2} \cdot {Tr}_{fiber - {∞}_{cal}}^{2} - {I}_{lens - {0}_{cal}} )\end{matrix} )} ] \} \} \cdot \\ \frac{1}{{Tr}_{{lens}_{low}}^{2} \cdot {Tr}_{{blast}_{low}}^{2} \cdot {Tr}_{{fiber}_{low}}^{2}}\end{matrix}}{{I}_{DET - {ref}_{low}}}} )}{2 ( {α}_{low} - {α}_{high} )} \qquad Equation 6.11$$

As outlined above, the configuration shown in FIG. 15, or rather the LETD system 1500, is arranged to “snatch” light reflected of the proximal end 202 of the optical fiber 104 to calibrate the optical components of the system (e.g., focus lens 1514, blast shield 1512, etc.) In general, light reflection 1510 can be represented by Equation 6.12 shown below, wherein f_{λ} is the factor of the signal going out in the direction of the light snatching, Isig_{λ} is the signal defined in

I_{snatched}_{_{λ}}=f_{λ}*(I_{sig}_{_{λ}}+I_{dist}_{_{λ}})+Iₚᵣₒₓ_{_{λ}}: Equation 6.12

Equation 6.12 can be rewritten into the polarized form as shown in Equation 6.13, where [symbol] is a mixed polarization value, [symbol] is an onward source polarized value, and [symbol] is perpendicular to source polarized value.

$$\overleftrightarrow{{I}_{{snatched}_{λ}}} = [ {f}_{λ} * {Tr}_{{fiber}_{λ}}^{2} * ( \vec{{I}_{{sig}_{λ}} + {I}_{{dist}_{λ}}} ) + \vec{{I}_{{prox}_{λ}}} ] + [ {f}_{λ} * {Tr}_{{fiber}_{λ}}^{2} * \overleftarrow{( {I}_{{sig}_{λ}} + {I}_{{dist}_{λ}} )} ] \qquad Equation 6.13$$

With distance at, ∞, this equation is reduced to Equation 6.14.

$$\overleftrightarrow{{I}_{snatched - {∞}_{λ}}} = [ {f}_{λ} * {Tr}_{{fiber}_{λ}}^{2} * \vec{{I}_{dist - {∞}_{λ}}} + \vec{{I}_{prox - {∞}_{λ}}} ] + {f}_{λ} * {Tr}_{{fiber}_{λ}}^{2} * \overleftarrow{{I}_{dist - {∞}_{λ}}} \qquad Equation 6.14$$

Using the value of I_{dist-∞}_{_{λ}} from the above equations, f_{λ}*Tr_{fiber}_{_{λ}}² can be extracted from the calibration signals. Accordingly, in the “snatched” light signal (e.g., light reflection 1510) the proximal end 202 reflection is Iₚᵣₒₓ_{_{λ}}=r_{snatchedλ}*Trₗₑₙₛ_{_{λ}}²*Tr_{blast}_{_{λ}}²*I_{DET-ref}_{_{λ}} so r_{snatchedλ}*Trₗₑₙₛ_{_{λ}}²*Tr_{blast}_{_{λ}}² is calibrated. As a result, any change in the values of r_{snatchedλ}*Trₗₑₙₛ_{_{λ}}²*Tr_{blast}_{_{λ}}² can be translated into Equation 6.7 per wavelength. The value of Iₗₑₙₛ_{_{λ}} mentioned above will accordingly be, be Iₗₑₙₛ_{_{λ}}=I_{DET-ref}_{_{λ}}*(1−Trₗₑₙₛ_{_{λ}}), so changes in r_{snatchedλ}*Trₗₑₙₛ_{_{λ}}²*Tr_{blast}_{_{λ}}² in the square root will compensate for these movements as well.

It will be appreciated that it will be advantageous to calibrate Iₗₑₙₛ₋₀_{_{λ}} as well as at ∞ from the beginning of a procedure.

Further, where polarized light is used, noise in the polarization switch (e.g., WDM 704, or the like) can be accounted for using Equation 6.15.

$$X = \frac{ln ( \frac{\begin{matrix}\frac{( \begin{matrix}{I}_{high} * [ {pol}_{{switch}_{{in}_{fiber}}} * {pol}_{transmission} + \\ ( 1 - {pol}_{{switch}_{{in}_{fiber}}} ) * ( {pol}_{transmission} + {pol}_{{transmission}_{diff}} ) ]\end{matrix} )}{{I}_{ref ( h )}} - \\ {I}_{ref - norm ( h ) - st} - {I}_{ref - norm ( h ) - d}\end{matrix}}{{I}_{low} / {I}_{ref ( l )} - {I}_{ref - norm ( l ) - st} - {I}_{ref - norm ( l ) - d}} )}{( 2 ( {λ}_{low} - {λ}_{high} ) )} \qquad Equation 6.15$$

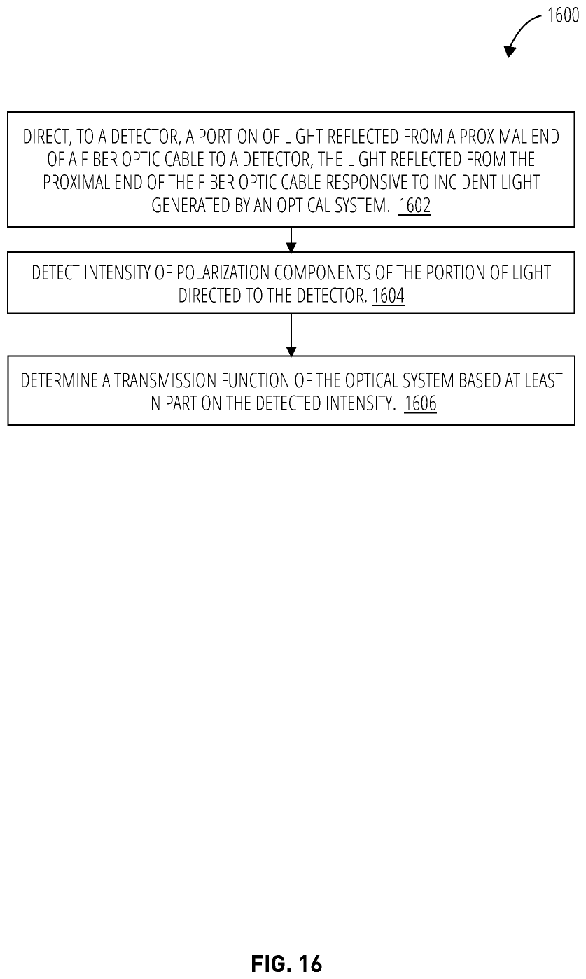

FIG. 16 illustrates a flowchart showing a method 1600 of estimating distance between a fiber end and a target in accordance with some embodiments of the present disclosure. The method 1600 is described with reference to the system 100 and to the configurations of the LETD system 106 described above with respect to FIG. 15, or rather, LETD system 1500. It is to be appreciated however, that the method 1600 could be implemented using an LETD system different than that described herein. Embodiments are not limited in this context.

At block 1602, mirror 1502 directs a portion of light reflected from the proximal end 202 of the optical fiber 104 to polarizing beam splitter 1504 and light detectors 1506a and 1506b. That is, mirror 1502 is arranged to direct a portion of light reflection 1510 to polarizing beam splitter 1504. Continuing to block 1604, method 1600 can detect an intensity of polarization components of the portion of light directed to the detector. In particular, light reflection 1510 is split into polarization components by polarizing beam splitter 1504 and light detectors 1506a and 1506b can detect an intensity of the components of the light reflection 1510.

At block 1606, method 1600 can determine a transmission function of the optical system based at least in part on the intensity detected at block 1604. For example, processing unit 108 can execute instructions to determine a transmission of the optical system as outlined above with Equations 6.1 to 6.17.

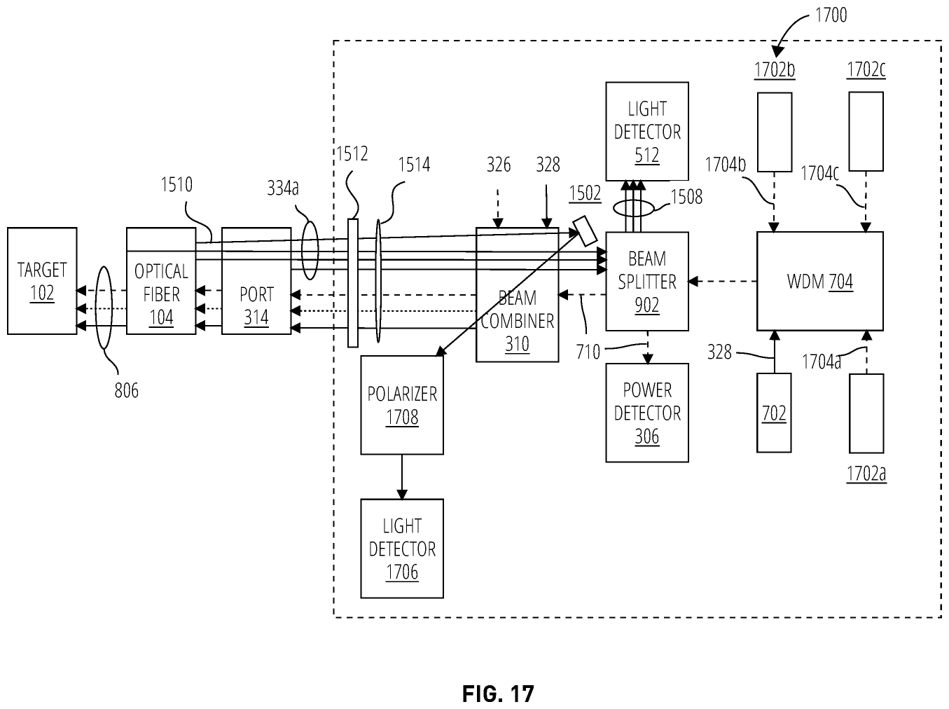

FIG. 17 illustrates a LETD system 1700, which is like the LETD system 1500 but with a single 1706 and polarizer 1708 as opposed to the polarizing beam splitter 1504 and light detectors 1506a and 1506b. Further, it is noted that LETD system 1700 can be implemented where the light laser sources 1702a, 1702b, and 1702c are arranged to emit non-polarized light beams 1704a, 1704b, and 1704c, respectively. Processing unit 108 can be arranged to account for changes in the optical components over time as described above with respect to FIG. 15 and FIG. 16, with the note that the Equations for the transmission function will not depend on polarization as outlined above.

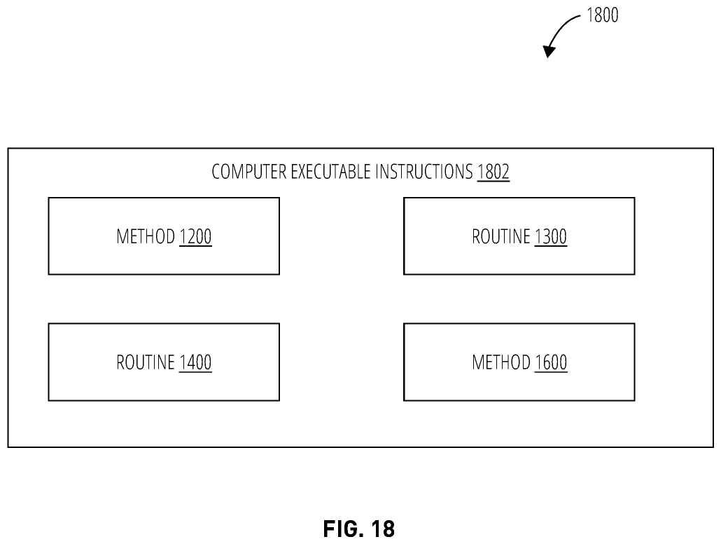

FIG. 18 illustrates computer-readable storage medium 1800. Computer-readable storage medium 1800 may comprise any non-transitory computer-readable storage medium or machine-readable storage medium, such as an optical, magnetic or semiconductor storage medium. In various embodiments, computer-readable storage medium 1800 may comprise an article of manufacture. In some embodiments, computer-readable storage medium 1800 may store computer executable instructions 1802 with which circuitry (e.g., processing unit 108, or the like) can execute. For example, computer executable instructions 1802 can include instructions to implement operations described with respect to method 1200, method 1300, method 1400, and/or method 1600. Examples of computer-readable storage medium 1800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1802 may include any suitable type of code, such as source code, compiled code, interpreted code, executable code, static code, dynamic code, object-oriented code, visual code, and the like.

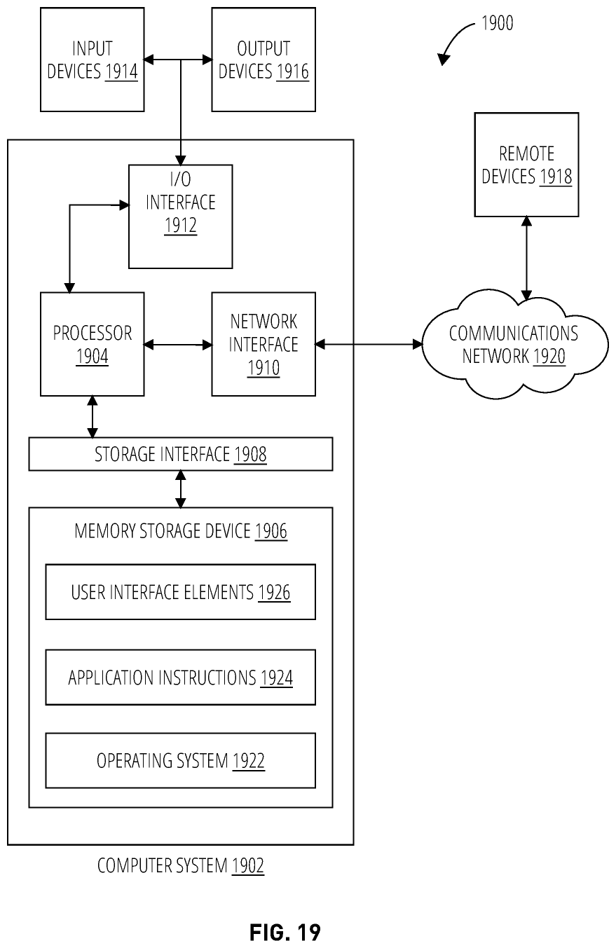

FIG. 19 is a block diagram of a computing environment 1900 including a computer system 1902 for implementing embodiments consistent with the present disclosure. In some embodiments, the computing environment 1900, or portion thereof (e.g., the computer system 1902) may comprise or be comprised in a laser system (e.g., the system 100, LETD system 106, etc.). Accordingly, in various embodiments, computer system 1902 may be used to determine a distance between a distal end 204 and an optical fiber 104 and a target 102 and account for changes of the optical system in time as outlined above.

The computer system 1902 may include a central processing unit (“CPU” or “processor”) 1904. The processor 1904 may include at least one data processor for executing instructions and/or program components for executing user or system-generated processes. A user may include a person, a person using a device such as those included in this disclosure, or another device. The processor 1904 may include specialized processing units such as integrated system (bus) controllers, memory management control units, floating point units, graphics processing units, neural processing units, digital signal processing units, etc. The processor 1904 may be disposed in communication with input devices 1914 and output devices 1916 via I/O interface 1912. The I/O interface 1912 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 1912, computer system 1902 may communicate with input devices 1914 and output devices 1916. In some embodiments, the processor 1904 may be disposed in communication with a communications network 1920 via a network interface 1910. In various embodiments, the communications network 1920 may be utilized to communicate with a remote memory storage device 1906, such as for accessing look-up tables, performing updates, or utilizing external resources. The network interface 1910 may communicate with the communications network 1920. The network interface 1910 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 1920 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 1920 may include a variety of network devices, including routers, bridges, servers, computing devices, storage devices, etcetera. In some embodiments, the processor 1904 may be disposed in communication with a memory storage device 1906 via a storage interface 1908. The storage interface 1908 may connect to memory storage device 1906 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 1906 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 1906 may store a collection of program or database components, including, without limitation, an operating system 1922, an application instructions 1924, and a user interface elements 1926. In various embodiments, the operating system 1922 may facilitate resource management and operation of the computer system 1902. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM® OS/2®, MICROSOFT® WINDOWS® (XP®, VISTA®/7/8, 10 etc.), APPLE® IOS®, GOOGLE™ ANDROID™, BLACKBERRY® OS, or the like.

The application instructions 1924 may include instructions that when executed by the processor 1904 cause the processor 1904 to perform one or more techniques, steps, procedures, and/or methods described herein, such to irrigate a site and irradiate a site as outlined herein. For example, application instructions 1924, when executed by processor 1904 can cause processor 1904 to perform the method 1200, method 1300, method 1400, and/or method 1600.

The user interface elements 1926 may facilitate display, execution, interaction, manipulation, or operation of program components through textual or graphical facilities. For example, user interfaces may provide computer interaction interface elements on a display system operatively connected to the computer system 1902, such as cursors, icons, checkboxes, menus, scrollers, windows, widgets, etcetera. The user interface elements 1926 may be employed by application instructions 1924 and/or operating system 1922 to provide, for example, a user interface with which a user can interact with computer system 1902. In some embodiments, the user interface elements 1926 may be integrated with the display (not shown).

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A system, comprising:
a first laser source to generate laser light of a first wavelength;
a second laser source to generate laser light of a second wavelength;
an optical fiber having a distal end and a proximal end, the optical fiber configured to receive laser light from the first and second laser sources at the proximal end, to reflect a portion of the laser light from the proximal end, to emit a portion of the laser light out of the distal end, and to receive reflected laser light into the distal end;
a first light detector to measure intensity of the reflected light;
a second light detector;
a mirror to direct, to the second light detector, the portion of the laser light reflected from the proximal end, the second light detector to measure intensity of the portion of the laser light reflected from the proximal end; and
a processor and memory comprising instructions that when executed by the processor cause the processor to estimate a distance between the distal end of the optical fiber and a target based on the intensity of the reflected light measured by the first light detector and the intensity of the portion of the laser light reflected from the proximal end measured by the second light detector.

2. The system of claim 1, wherein the first wavelength has a first water absorption coefficient higher than a second water absorption coefficient of the second wavelength.

3. The system of claim 2, wherein the ratio of the first water absorption coefficient to the second water absorption coefficient is at least 2 to 1.

4. The system of claim 3, wherein the first wavelength is approximately 1330 nm to approximately 1380 nm and the second wavelength is approximately 1260 nm to approximately 1320 nm.

5. The system of claim 4, comprising a third laser source to generate laser light of a third wavelength utilized to characterize a condition of the optical fiber, wherein the third wavelength has a third water absorption coefficient higher than the first and the second water absorption coefficients.

6. The system of claim 5, wherein the third wavelength comprises approximately 1435 nm, approximately 2100 nm, or a wavelength between approximately 1870 nm and approximately 2050 nm.

7. The system of claim 1, wherein the light detector measures a first intensity value of the reflected light corresponding to the laser light of the first wavelength and a second intensity value of the reflected light corresponding to the laser light of the second wavelength.

8. The system of claim 7, wherein the instructions, when executed by the processor, further cause the processor to:
compute a ratio of the first intensity value and the second intensity value; and
estimate the distance between the distal end of the optical fiber and the target based on the ratio of the first intensity value and the second intensity value.

9. The system of claim 1, wherein one or more of the first and second laser sources comprise a polarization maintaining pigtailed fiber laser, a single mode pigtailed fiber laser, or a free space laser.

10. The system of claim 1, comprising a wave division multiplexer (WDM) coupled to a proximal end of the optical fiber, the WDM to arrange the laser light of the first wavelength and the laser light of the second wavelength to enter a proximal end of the optical fiber at one or more of a same point and a same angle.

11. At least one non-transitory computer-readable medium comprising a set of instructions that, in response to being executed by a processor circuit, cause the processor circuit to:
determine a first intensity values based on first reflected laser light corresponding to a laser source, wherein the first reflected laser light enters a proximal end of an optical fiber, exits a distal end of the optical fiber, reflects of a target, and enters the distal end of the optical fiber;
determine a second intensity value based on second reflected laser light corresponding to the laser source, wherein the second reflected laser light is reflected of the proximal end of the optical fiber;
compute a ratio of the first intensity value and the second intensity value; and
estimate a distance between the distal end of the optical fiber and the target based on the ratio of the first intensity value and the second intensity value.

12. The at least one non-transitory computer-readable medium of claim 11, wherein the set of instructions, in response to execution by the processor circuit, further cause the processor circuit to subtract a first internal reflection value from a first measured intensity value to determine the first intensity value and subtract a second internal reflection value from a second measured intensity value to determine the second intensity value.

13. The at least one non-transitory computer-readable medium of claim 11, wherein the set of instructions, in response to execution by the processor circuit, further cause the processor circuit to determine an internal reflection value based on third reflected laser light corresponding to laser light of a third wavelength, wherein the laser light of the third wavelength exits a laser source and the at least a portion of the third reflected laser light is reflected by a distal end of the optical fiber.

14. The at least one non-transitory computer-readable medium of claim 13, wherein the set of instructions, in response to execution by the processor circuit, further cause the processor circuit to:
compare the internal reflection value to a baseline internal reflection value; and
adjust an operating parameter of a treatment beam based on comparison of the internal reflection value to the baseline internal reflection value.

15. The at least one non-transitory computer-readable medium of claim 14, wherein the set of instructions, in response to execution by the processor circuit, further cause the processor circuit to:
compare the internal reflection value to a baseline internal reflection value;
characterize a condition of the optical fiber based on comparison of the internal reflection value to the baseline internal reflection value; and
communicate an indication of the condition of the optical fiber via a user interface.

16. The at least one non-transitory computer-readable medium of claim 11, wherein the set of instructions, in response to execution by the processor circuit, further cause the processor circuit to communicate an indication of the distance estimated between the distal end of the optical fiber and the target via a user interface.

17. A method, comprising:
illuminating a target with laser light of a plurality of different wavelengths;
receiving first reflected light beams from the target via an optical fiber;
receiving second reflected light beams from a proximal end of the optical fiber;
measuring intensity of the first and second reflected light beams with a plurality of light detectors;
estimating a distance between a distal end of the optical fiber and the target based on intensity of the reflected light beams measured with the one or more light detectors;

18. The method of claim 17, comprising emitting the laser light of the plurality of different wavelengths via the optical fiber to illuminate the target.

19. The method of claim 17, comprising measuring a first intensity value of the reflected light beams corresponding to laser light of a first wavelength and a second intensity value of the reflected light beams corresponding to laser light of a second wavelength.

20. The method of claim 19, comprising:
computing a ratio of the first intensity value and the second intensity value; and
estimating the distance between the distal end of the optical fiber and the target based on the ratio of the first intensity value and the second intensity value.
